(19) United States
(12) Patent Application Publication
(10) Pub. No.: US 2016/0120975 A1
(43) Pub. Date: May 5, 2016

(54) FISH VACCINE

(71) Applicant: THE UNIVERSITY COURT OF THE UNIVERSITY OF ABERDEEN, Aberdeen, Aberdeenshire (GB)

(72) Inventors: Stephan WAWRA, Aberdeen (GB); Pieter VAN WEST, Aberdeen (GB); Christopher John SECOMBES, Aberdeen (GB)

(21) Appl. No.: 14/894,659

(22) PCT Filed: May 30, 2014
(86) PCT No.: PCT/GB2014/051655

§ 371 Date: Nov. 30, 2015

(30) Foreign Application Priority Data
May 30, 2013 (GB) 1309664.9

(51) Int. Cl. A61K 39/385; A61K 9/08; C07K 14/44

(52) U.S. Cl. CPC A61K 39/385; C07K 14/44; A61K 9/08; A61K 2039/57

(57) ABSTRACT

A composition comprising a translocation sequence derived from a fish pathogen and a heterologous payload coupled to the translocation sequence is provided. The composition is able to translocate across the plasma membrane of a eukaryotic cell, for example a fish cell, and thus stimulate an immune response. Accordingly, nucleic acids, vectors, host cells, compositions and vaccines based thereon are also provided.

The present invention relates to vaccine compositions for fish.

### BACKGROUND

The farming of fish, molluscs and crustaceans plays an important and growing part in the global supply of food. Farmed fish is supplying ˜30% by weight of world total fish production in 2001 (up from ˜4% in 1970). Aquaculture is therefore an important industry, with the culture of high-value species such as salmon and salmon trout worth US$10.7 billion in 2007.

Atlantic salmon is a particularly important species, and is the most intensively farmed fish in the world. Major producers of farmed Atlantic salmon include Norway, Chile, Canada, and the UK.

Maintaining the health of farmed fish stocks is crucial for maximizing fish survival rate, growth and therefore profitability in aquaculture. Controlling the levels of fish borne diseases and parasites are key to this.

In the wild fish populations, diseases and parasites are typically present at low levels. Transmission of pathogens is reduced in the relatively low population densities seen in wild stocks, with numbers of sick individuals also kept in check by natural predation. However, in the crowded net pens commonly used in aquaculture, the farmed fish are shielded from natural predators and kept in close proximity to infected individuals. Under these conditions, diseases and parasites can rapidly enter the farmed population and reach epidemic levels.

Disease control in Aquaculture

In order to control the levels of disease, farmed fish can be vaccinated against the most common pathogens which affect farmed fish. The advent of widespread vaccination has significantly boosted the productivity of aquaculture, as well as allowing the use of antibiotics to be significantly reduced.

Vaccination Techniques

Vaccines against a broad range of pathogenic antigens are available, including those from Vibrio anguillarum, Vibrio salmonicida, Aeromonas salmonicida, Yersinia ruckeri, Renibacterium salmonis, Piscirickettsia salmonis, IPN, IHN and ISA (see Sommerset et al. 2005, Expert Rev. Vaccines 4(1), 89-101). Fish may be vaccinated by oral administration, immersion (bath, dip or spray) or injection (intraperitoneal or intramuscular).

Much effort has been put into the development of oral vaccines, as this administration method offers the advantages of being easy to deliver (for example, in food), thereby saving labour on behalf of the farmer and minimizing stress to the fish. However, immunization by this route has been reported to be weak and of short duration, as well as requiring a large quantity of antigen to be delivered. These disadvantages mean that there has been little uptake of oral vaccines (see Sommerset et al. 2005, Expert Rev. Vaccines 4(1), 89-101).

Immersion vaccines share many of the features of oral vaccines: they are easy to deliver and minimize the stress to the fish, but also typically require large amounts of antigen and result in immunity that is weaker and of a shorter duration than that offered by injection. However, unlike oral administration, there are several pathogens for which immersion vaccinations are considered to offer reasonable protection (for example, versus infections by Vibrio anguillarum, Vibrio salmonicida and Yersinia ruckeri).

WO2011/148135 describes proteins and protein constructs and their translocation into cultured the fish cell lines RTG-2 (trout gonad), RTL (trout liver), RTGill (trout gill) and SDJ (Zebra fish).

Despite the availability of effective immersion vaccines against some pathogens, many of the economically important diseases (such as furunculosis) require that the commercially available antigen is delivered together with an adjuvant for an effective level of immunity is to be reliably stimulated. Since the available fish adjuvants are often oil-based, this means that antigens requiring an adjuvant cannot be delivered by the immersion method. Oral delivery is also often ineffective, presumably due to digestion of the antigen/adjuvant before an immune response is raised.

A consequence of the adjuvant requirement by economically important disease antigens is that vaccines for these diseases must be injected (for example, intraperitoneally or intramuscularly). Since a single injection can be used to deliver more than one type of antigen, this means that ‘multi-antigen injected vaccines’ are currently the vaccination method of choice in the aquaculture industry (see Sommerset et al. 2005, Expert Rev. Vaccines 4(1), 89-101).

Vaccination by injection offers the advantage that it reliably generates a strong and long-lasting immune response. However, the injection process is costly and labour-intensive and causes significant handling stress on the fish. This creates a risk of post-vaccination infection (in particular, Saprolegnia infections), which can decrease fish survival and growth rates.

In addition to the risk of post-handling infection, injection can also lead to so-called “local reactions” in the injected fish. These “local reactions” include, for example, local and/or diffuse peritonitis with adhesions to internal organs adhesions, melanisation, and/or multiple granulomata. It is believed that in some cases, the use of adjuvants with the injected antigen increases the severity of these reactions. As well as being a fish welfare issue, these reactions can lead to reduced growth rates, fish that are unfit for consumption and interfere with fish processing (such as gutting).

In view of the above discussion of existing vaccination methods, there is an ongoing need for alternative fish vaccination methods that avoid the disadvantages of injection/adjuvants but which still reliably deliver high levels of long-lasting immunity.

### DISCLOSURE OF THE INVENTION

The present inventors have cloned and expressed a fusion protein containing an antigenic polypeptide (RFP) fused to amino acids 24 to 68 of the protein SpHtp1, a putative effector from the fish pathogenic oomycete, Saprolegnia parasitica. Rainbow trout (Oncorhynchus mykiss) were then exposed to the composition by immersing the trout in a dilute solution (3 μM) of the composition for a short time (1.5 hours). Surprisingly, and as shown in the examples, the composition was able to enter the cells of the live fish and elicit an immune (antibody) response against the antigenic polypeptide. No adjuvants were required to stimulate the immune response. Injection of the composition into the fish was not required.

Thus, the findings of the present inventors allow for the effective vaccination of fish against almost any antigen without the need for injection and the resultant stress and infection risk to the fish. By coupling an antigen to the SpHtp1 translocation sequence and immersing a fish in a solution of the resulting composition, an immune response against the antigen can be stimulated.

Thus, in one aspect of the invention, the invention provides a composition comprising, consisting, or consisting essentially of: (i) a translocation sequence; and (ii) a heterologous payload coupled to the translocation sequence.

The properties of the translocation sequence enable the composition to translocate across the plasma membrane of a eukaryotic cell, for example a fish cell. In some embodiments the composition is able to translocate across the plasma membrane of cells in culture (for example, a RTG-2 cell line, a RTL cell line, a RTGill cell line, or a RTS11cell line) and/or across the plasma membrane of cells in a live fish (for example, cells present in the gills or gut of the Rainbow trout (Oncorhynchus mykiss) and other species important for marine, brackish and fresh water aquaculture such as salmonids, catfish, carps, sea bass, flat fish, and Tilapia's. In particular: Grass carp (Ctenopharyngodon idella), Silver carp (Hypophthalmichthys molitrix), Catla (Cyprinus catla or Gibelion catla), Common Carp (Cyprinus carpio), Bighead carp (Hypophthalmichthys nobilis or Aristichthys nobilis), Crucian carp (Carassius carassius), Oreochromis niloticus Nile Tilapia (Oreochromis niloticus), Mozambique Tilapia (Oreochromis mossambicus) and other Tilapia's, Pangas catfishes (Pangasius pangasius), Roho (Labeo rohita), Atlantic salmon (Salmo salar), Arctic charr (Salvelinus alpinus), brown trout (Salmo trutta), rainbow trout (Oncorhynchus mykiss), sea trout (Salmo trutta) and other trouts. In some embodiments the composition stimulates an immune (e.g. antibody) response in an organism (e.g. fish) that is exposed to the composition; preferably, the stimulated antibodies recognize an epitope in the payload (which may be, for example, a peptide antigen).

As described herein, the translocation sequence may comprise, consist or consist essentially of a polypeptide having the sequence of SpHtp1²⁴⁻⁶⁸ or SpHtp1a²⁴⁻⁶⁹ as shown in FIG. 1. As shown in the examples, this polypeptide is capable of directing a heterologous payload into the cells of a live fish such that the fish mounts an immune (antibody) response against the payload.

The invention further provides nucleic acids, vectors and host cells suitable for production of the composition of the invention, along with vaccines comprising the composition.

Also provided herein are processes for producing the nucleic acids, vectors, host cells, compositions and vaccines based thereon.

Some aspects and embodiments of the present invention will now be discussed in more detail.

Translocation Sequence

Thus, in one aspect the compositions of the invention comprise a translocation sequence which comprises, consists or consists essentially of a polypeptide having the sequence of SpHtp1²⁴⁻⁶⁸ or SpHtp1a²⁴⁻⁶⁹ as shown in FIG. 1.

Origin of SpHtp1

SpHtp1 is a putative effector from the fish pathogenic oomycete, Saprolegnia parasitica.

The inventors have previously shown that the host targeting protein SpHtp1 bind to tyrosine-O-sulfate, and that sulfatase treatment of cells strongly decreases the observed uptake of saprolegnia host targeting protein (works also with SpHtp1a) proteins into those cells (Wawra et al., 2012). It is therefore believed that these proteins and homologs thereof recognize and bind O-sulfated cell surface molecules as part of the translocation mechanism.

The amino acid sequences of ‘full-length’ SpHtp1 (1-198) and a number of fragments are shown in FIG. 1.

Earlier work of the inventors has shown that a SpHtp1²⁴⁻⁶⁸:RFP composition is able to enter cultured fish cells; translocation has been observed into trout epithelia cells (RTG-2 cell line) (see Wawra et al. 2012, www.pnas.org/cgi/doi/10.073/pnas.1113775109), trout liver cells (RTL cell line), trout gill cells (RTGill cell line) (Submitted paper Lobach and Wawra et al.), the macrophage cell line RTS11 and primary macrophages and a Zebra fish cell line (SDJ.1). This process can be inhibited by e.g. the peanut agglutinin and soy bean agglutinin in an allosteric manner on all tested fish cell lines (RTG-2, RTL, RTGill).

Notwithstanding the above, the novel finding described herein that the SpHtp1²⁴⁻⁶⁸:RFP composition can translocate into the cells of live fish immersed in a dilute solution of the composition (and, thereby, elicit an immune response) is unexpected. Cells in culture are relatively ‘open’, that is, the surrounding media is relatively benign and their surface membranes are relatively accessible; in comparison, tissues in live fish that are exposed to the external environment (e.g. gut, gills, skin) have defences such as a mucous layer and extracellular/surface proteases designed to prevent any unwanted exogenous molecules from entering the fish. Accordingly, the fact that the SpHtp1²⁴⁻⁶⁸:RFP composition was able to survive the ‘live’ environment, enter the fish, and induce an immune response was highly surprising.

The ability of the SpHtp1′ composition to cross cell membranes appears to be fish-specific, since the same composition was not able to translocate into human (HEK293) or onion cells (see Wawra et al. 2012, www.pnas.org/cgl/doi/0.1073/pnas.1113775109) or the human A549 cell line (Wawra et al. 2012, MPMI Vol 26 (5) pp 528-36). However, the reported ability of some other proteins to enter non-fish cells suggest that an amino acid variant of the SpHtp1²⁴⁻⁶⁸ composition may have a different, or no, organism specificity.

SpHtp1 as a Translocation Sequence

‘Full-length’ SpHtp1 contains 198 amino acids (see FIG. 1). However the fragment consisting of amino acids 24 to 68 (SpHtp1²⁴⁻⁶⁸) fused to mRFP retains the ability to translocate into live fish cells. The SpHtp1 homolog, SpHtp1a, also has this translocation ability. Thus, in some embodiments the translocation sequence consists of, or consists essentially of, SpHtp1²⁴⁻⁶⁸ or SpHtp1a²⁴⁻⁶⁹ shown in FIG. 1.

Longer polypeptides comprising the SpHtp1²⁴⁻⁶⁸ or SpHtp1a²⁴⁻⁶⁹ fragments have the ability to translocate into the cells of live fish. Thus, in some embodiments the translocation sequence comprises SpHtp1²⁴⁻⁶⁸ or SpHtp1a²⁴⁻⁶⁹ shown in FIG. 1. The translocation sequence may be of any length. However, in some embodiments it is no more than 200 amino acids, such as no more than 150, no more than 125, no more than 100 or no more than 75 amino acids.

In some embodiments the translocation sequence is no more than 60 amino acids. In some embodiments the translocation sequence is no more than 50 amino acids.

Where the SpHtp1²⁴⁻⁶⁸ or SpHtp1a²⁴⁻⁶⁹ shown in FIG. 1 forms part of a longer translocation sequence, the SpHtp1²⁴⁻⁶⁸ or SpHtp1a²⁴⁻⁶⁹ fragment can be at any point of the translocation sequence: at the very N-terminal, at the very C-terminal, or at an intermediate point between the N- and C- terminals.

Variants

Sequence variants of SpHtp1²⁴⁻⁶⁸ are also able to translocate into the cells of live fish. Thus, in some embodiments the translocation sequence consists of, or consists essentially of, a sequence variant of SpHtp1²⁴⁻⁶⁸ shown in FIG. 1. For example, the SpHtp1 homolog SpHtp1a shown in FIG. 1 is able to translocate into the cells of live fish. Thus, in some embodiments the translocation sequence consists of, or consists essentially of, a sequence variant of SpHtp1a²⁴⁻⁶⁹ shown in FIG. 1.

Longer polypeptides comprising sequence variants of the SpHtp1²⁴⁻⁶⁸ or SpHtp1a²⁴⁻⁶⁹ shown in FIG. 1 have the ability to translocate into the cells of live fish. Thus, in some embodiments the translocation sequence comprises a sequence variant of SpHtp1²⁴⁻⁶⁸ or SpHtp1a²⁴⁻⁶⁹ shown in FIG. 1. The translocation sequence may be of any length. However, in some embodiments it is no more than 200 amino acids, such as no more than 150, no more than 125, no more than 100 or no more than 75 amino acids. In some embodiments the translocation sequence is no more than 60 amino acids. In some embodiments the translocation sequence is no more than 50 amino acids.

Where the sequence variant of SpHtp1²⁴⁻⁶⁸ or SpHtp1a²⁴⁻⁶⁹ shown in FIG. 1 forms part of a longer translocation sequence, the variant SpHtp1²⁴⁻⁶⁸ or SpHtp1a²⁴⁻⁶⁹ fragment can be at point of the translocation sequence: at the very N-terminal, at the very C-terminal, or at an intermediate point between the N- and C- terminals.

As used herein, a “sequence variant of SpHtp1²⁴⁻⁶⁸ or SpHtp1a²⁴⁻⁶⁹ shown in FIG. 1” is an amino acid sequence having at least 60, 70, 75, 80, 85, 90, 95, 96, 97, 98, 99 or 100% identity to the amino acid sequence SpHtp1²⁴′ or SpHtp1a²⁴⁻⁶⁹ shown in FIG. 1.

In one embodiment the translocation sequence will have equal to, or no more than, 1, 2, 3, 4, 5, 6, 7, 8, 9, 10, 12, 15 or 20 substitutions, deletions, or additions in the amino acid sequence SpHtp1²⁴⁻⁶⁸ or SpHtp1a²⁴⁻⁶⁹ as shown in FIG. 1.

Identity may be as defined using sequence comparisons are made using FASTA and FASTP (see Pearson & Lipman, 1988. Methods in Enzymology 183: 63-98). Parameters are preferably set, using the default matrix, as follows: Gapopen (penalty for the first residue in a gap): −12 for proteins/−16 for DNA; Gapext (penalty for additional residues in a gap): −2 for proteins/−4 for DNA; KTUP word length: 2 for proteins/6 for DNA.

For the avoidance of doubt, the level of sequence identity is measured over the full-length of the amino acid sequence SpHtp1²⁴⁻⁶⁸ or SpHtp1a²⁴⁻⁶⁹ shown in FIG. 1 (45 or 46 amino acids).

The variant translocation sequences may originate from other native proteins, or may be prepared by those skilled in the art, for instance by site directed or random mutagenesis of the SpHtp1²⁴⁻⁶⁸ or SpHtp1a²⁴⁻⁶⁹ sequence shown in FIG. 1. Alternatively the variant translocation sequences maybe produced by direct synthesis.

Changes may be desirable for a number of reasons, including introducing or removing the following features: sites which are required for post translation modification; cleavage sites in the encoded polypeptide; motifs in the encoded polypeptide (e.g. epitopes). Leader or other targeting sequences (e.g. hydrophobic anchoring regions) may be added or removed from the expressed protein to determine its location following expression.

Other desirable mutations may be made by random or site directed mutagenesis of the nucleic acid encoding the polypeptide in order to alter the activity (e.g. specificity) or stability of the encoded polypeptide.

Changes may be by way of conservative variation, i.e. substitution of one hydrophobic residue such as isoleucine, valine, leucine or methionine for another, or the substitution of one polar residue for another, such as arginine for lysine, glutamic for aspartic acid, or glutamine for asparagine. As is well known to those skilled in the art, altering the primary structure of a polypeptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptides conformation.

Also included are variants having non-conservative substitutions. As is well known to those skilled in the art, substitutions to regions of a peptide which are not critical in determining its conformation may not greatly affect its ability to raise antibodies because they do not greatly alter the peptide's three dimensional structure.

In regions which are critical in determining the peptides conformation or activity such changes may confer advantageous properties on the polypeptide. Indeed, changes such as those described above may confer slightly advantageous properties on the peptide e.g. altered specificity, stability or immunogenicity.

Payload

The payload is a “heterologous payload”. As used herein, a “heterologous payload” is a payload that is heterologous to the translocation sequence to which it is coupled. In this context, “heterologous” means that the translocation sequence and payload are not derived from the same organism (for example, not from the same strain or species); that is, the translocation sequence and payload are derived from different organisms (for example, derived from different strains or species). Accordingly, in embodiments where the translocation sequence is SpHtp1²⁴⁻⁶⁸ or SpHtp1a²⁴⁻⁶⁹ shown in FIG. 1, the payload is not from Saprolegnia parasitica (the organism in which SpHtp1 and SpHtp1a are natively found).

The payload may be any type of molecule, for example a polypeptide, nucleic acid, lipid or small organic molecule. The terms “peptide”, “polypeptide” and “protein” as used herein are used interchangeably to mean a polymer of two or more amino acids coupled through peptide bonds.

Preferably, the payload is a peptide or polypeptide. In embodiments where the payload is a peptide or polypeptide, the translocation sequence may be of any length. However, in some embodiments it is no more than 500 amino acids, such as no more than 400, no more than 300, no more than 200 or no more than 100 amino acids. In some embodiments the translocation sequence is no more than 75 amino acids. In some embodiments the translocation sequence is no more than 50 amino acids, such as no more than 30 amino acids.

In some embodiments the payload is a particle or bead. For example, the payload may be a particle with high contrast in detection methods (such as a gold particle).

In some embodiments the payload is a therapeutic agent, a marker (e.g. a fluorescent protein such as GFP or RFP) or a protective agent (e.g. an agent that protects the cell from the effects of a cytotoxin to which the cell is subsequently exposed). In some embodiments the payload is a cytotoxic molecule (i.e. a molecule, which when bound to or taken up by a target cell stimulates the death and lysis of the cell) or an RNA molecule such as a siRNA.

Cytotoxic molecules include members of the following groups or families: nitrogen—mustard types (e.g. melphalan), anthracyclines (e.g. adriamycin, doxorubicin, and daunomycin), nucleoside analogues (e.g. cytosine arabinoside) or antimetabolites (e.g. methotrexate). Also encompassed by the term “cytotoxic molecules” as used herein are enzymes intended to catalyse the conversion of a non-toxic prodrug into a cytotoxic drug (for example a HSV-Thymidine Kinase/Ganciclovir system). The prodrug may be systemically administered.

In some embodiments the payload comprises an antigen or immunogen.

Antigens and Immunogens

In some embodiments the payload consists of a single antigen. In other embodiments, the payload comprises one, two, three, four, five, six, seven, eight, or more than eight antigens.

In embodiments where the payload comprises more than one antigen, each antigens can be from the same or different organisms. In one embodiment, each antigen in the payload is from a different organism (i.e. antigens 1, 2, 3, 4 are correspondingly form organisms A, B, C, D).

As used herein, the term “antigen” is used to describe a substance that provokes the production of one or more antibodies when introduced into an organism (as detectable by techniques such as ELISA).

An antigen may be a peptide, lipid or nucleic acid. Preferably, the antigen is a peptide antigen.

The antigen may be a specific type or group of molecule. For example, the antigen may be a viral coat protein, a viral envelope protein, a viral lipid, a viral glycan, a bacterial envelope protein, a bacterial coat protein, a bacterial lipid, and/or a bacterial glycan.

An antigen may contain only a single epitope. Alternatively, in some embodiments an antigen contains two, three, four, five or more than 5 epitopes.

The antigen may be from a fish pathogen. For example, the antigen may be a peptide or polypeptide from a fish pathogen. Example fish pathogens from which antigens may be from are (i) bacterial pathogens, for example Vibrio anguillarum & Vibrio salmonicida (vibriosis), Aeromonas salmonicida & Aeromonas hydrophila (furunculosis), Yersinia ruckeri (Enteric Redmouth Disease), Renibacterium salmonis, Lactococcus sp., Streptococcus sp., and Piscirickettsia salmonis; (ii) viral pathogens, for example Infectious pancreatic necrosis virus (IPNV), Infectious Salmon Anemia virus (ISAV), Salmon Pancreas Disease virus (SPD virus), Sleeping Disease of Trout virus (SDV), Viral Nervous Necrosis virus (VNNV) and Infectious Heamatopoietic Necrosis virus (IHNV), Viral haemorrhagic septicaemia virus (VHSV); (iii) Fungal pathogens, for example Saprolegnia sp. (such as S. parasitica, S. diclina and S. australis) or Aphanomyces sp. (A. invadans and A. astaci) and Branchiomyces sp. (Gill rot); and/or (iv) Parasitic pathogens, for example Ichthyophthirius multifiliis, Cryptocaryon irritans and Trichodina sp. (Trichodiniasis), Amoebic gill disease (eg. Neoparamoeba perurans), sea lice (copepods within the order Siphonostomatoida, family Caligidae (for example Lepeophtheirus salmonis, Caligus rogercresseyi, Caligus clemensi, Caligus elongatus), proliferative kidney disease or PKD (Tetracapsuloides bryosalmonae) and other species.

The antigen may be a peptide or polypeptide from a virus, for example a peptide or polypeptide from the outer proteins or coat proteins of a virus such as IPNV (example polypeptides, or encoding nucleotides=Genbank accession numbers ACY35988.1, ACY35989.1 ACY35990.1; UniProt accession numbers D0VF01, D0VF02, D0VF03), ISAV (example polypeptides, or encoding nucleotides =Genbank accession numbers EU625675, FJ594325; UniProt accession numbers C6ETL2, C6G756), SPD (example polypeptides, or encoding nucleotides =Genbank accession number AJ012631 .1 UniProt accession number Q9WJ34), SDV (example polypeptides, or encoding nucleotides=Genbank accession number AJ238578.1; UniProt accession number Q8QL52), IHNV (example polypeptides, or encoding nucleotides=Genbank accession number X89213.1; UniProt accession numbers Q08449, Q08455, Q08454, Q08453, Q82706, Q08445, Q08454, Q82707) VHSV (example polypeptides, or encoding nucleotides=Genbank accession numbers EU481506.1, ACA34520.1, ACA34521.1 ACA34522.1, ACA34523.1, ACA34524.1., ACA34525.1).

Coupling of Translocation Sequence and Payload

In some embodiments, the translocation sequence is covalently bonded to the payload, for example, via a peptide bond. In embodiments where the payload is a peptide or polypeptide the translocation sequence and the payload may be coupled via an peptide bond so that they form a contiguous polypeptide chain; that is, the composition of the invention may be a fusion protein comprising the translocation sequence fused to the peptide payload. The fusion protein of the invention may be of any length. However, in some embodiments it is no more than 500 amino acids, such as no more than 400, no more than 300, no more than 200 or no more than 100 amino acids. In some embodiments the translocation sequence is no more than 80 amino acids. In some embodiments the translocation sequence is no more than 60 amino acids.

The coupling of the translocation sequence and the payload may be direct i.e. without intervening elements (such as amino acids).

The payload may be an “exogenous protein” i.e. a protein that is not natively expressed in the eukaryotic cell into which it is being translocated. Exogenous proteins include, for example, fusions of native proteins, or fragments of native proteins, with non-native polypeptides or other molecules.

The translocation sequence and payload may be separated by a short linker sequence or residue in order to facilitate expression or folding e.g. one, two, three, four, five or more gly residues. However, the presence of a linker is not required. Similarly, in embodiments where the payload contains two or more antigens, the antigens may be separated by a short linker sequence or residue in order to facilitate expression or folding e.g. one, two, three, four, five or more gly residues. Again, the presence of a linker is not required.

Having described the fusion and other proteins of the present invention, some other aspects of the invention will now be discussed.

Nucleic Acids, Vectors And Host Cells

The compositions of the invention are conveniently prepared from recombinant nucleic acids which can be expressed in host cells with good yield, for example from a pET vector and can be expressed in Escherichia coli.

Thus in one aspect there is provided a nucleic acid encoding a composition of the invention. Such nucleic acid is herein referred to “composition nucleic acid” for brevity. This composition nucleic acid may be put together, for example, using “overlap PCR”.

Nucleic acid according to the present invention may include cDNA, RNA, genomic DNA and modified nucleic acids or nucleic acid analogues (e.g. peptide nucleic acid). Where a DNA sequence is specified, e.g. with reference to a figure, unless context requires otherwise the RNA equivalent, with U substituted for T where it occurs, is encompassed. Nucleic acid molecules according to the present invention may be provided isolated and/or purified from their natural environment, in substantially pure or homogeneous form, or free or substantially free of other nucleic acids of the species of origin, and double or single stranded. Where used herein, the term “isolated” encompasses all of these possibilities. The nucleic acid molecules may be wholly or partially synthetic. In particular they may be recombinant in that nucleic acid sequences which are not found together in nature (do not run contiguously) have been ligated or otherwise combined artificially. Nucleic acids may comprise, consist, or consist essentially of, any of the sequences discussed hereinafter.

Aspects of the invention further embrace isolated nucleic acid comprising a sequence which is complementary to any of those discussed hereinafter.

In one aspect of the present invention, the composition nucleic acid described above is in the form of a recombinant and preferably replicable vector.

Composition nucleic acids can be incorporated into a specially constructed vector or can be constituted in situ by introducing nucleic acid encoding the various portions such that the whole sequence is created in frame in the vector. Such processes for making the recombinant vectors form one aspect of the invention.

It is envisaged in the present invention that compositions as defined herein, based on newly characterized translocation sequences or payloads (e.g. from newly characterized antigens from new or known antigens) may be prepared simply and easily by sequencing, synthesis of DNA, followed by PCR and ligation (e.g. using the appropriate restriction sites).

“Vector” is defined to include, inter alia, any plasmid, cosmid, or phage in double or single stranded linear or circular form which may or may not be self-transmissible or mobilizable, and which can transform a prokaryotic or eukaryotic host either by integration into the cellular genome or exist extrachromosomally (e.g. autonomous replicating plasmid with an origin of replication).

Generally speaking, those skilled in the art are well able to construct vectors and design protocols for recombinant gene expression. Suitable vectors can be chosen or constructed, containing appropriate regulatory sequences, including transcriptional and translational regulatory elements, such as transcriptional enhancer sequences, translational enhancer sequences, promoters, ribosomal entry sites, including internal ribosomal entry sites, activators, translational start and stop signals, transcription terminators, cistronic regulators, polycistronic regulators, marker genes and other sequences as appropriate. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, 1992.

Specifically included are shuttle vectors by which is meant a DNA vehicle capable, naturally or by design, of replication in two different host organisms, which may be selected from actinomycetes and related species, bacteria and eukaryotic (e.g. yeast or fungal cells).

A vector including nucleic acid according to the present invention need not include a promoter or other regulatory sequence, particularly if the vector is to be used to introduce the nucleic acid into cells for recombination into the genome.

However preferably the nucleic acid in the vector is under the control of, and operably linked to, an appropriate promoter or other regulatory elements for transcription in a host cell such as a microbial, e.g. bacterial cell. By “promoter” is meant a sequence of nucleotides from which transcription may be initiated of DNA operably linked downstream (i.e. in the 3′ direction on the sense strand of double-stranded DNA). Example vectors include plasmids pQE, pET or other commercial or generally accessible plasmids.

A preferred vector is the pET11-a plasmid, although where expression is desired in XL1-blue, other vectors may be preferred.

“Operably linked” means joined as part of the same nucleic acid molecule, suitably positioned and oriented for transcription to be initiated from the promoter. DNA operably linked to a promoter is “under transcriptional initiation regulation” of the promoter.

In one embodiment, the promoter is an inducible promoter. The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus. A typical inducible promoter is the Iac promoter induced by IPTG.

The present invention also provides methods comprising introduction of such an expression construct into a cell e.g. a microbial cell (e.g. bacterial, yeast or fungal) cell or an insect cell and/or induction of expression of the expression construct within the cell, by application of a suitable stimulus e.g. an effective exogenous inducer.

In a further aspect of the invention, there is disclosed a host cell containing a expression construct according to the present invention, especially a microbial cell such as E. coli e.g. E. coli XL1-blue cells

The invention further encompasses a host cell transformed with nucleic acid or a vector according to the present invention (e.g. comprising the fusion nucleic acid) especially a microbial cell such as E. coli.

The fusion protein can be produced by culturing a microbial cell as described above e.g. by growing the cells in liquid medium with appropriate selection reagents (e.g. LB medium with ampicillin).

In another aspect of the invention provides a process for producing a composition for use in a vaccine in a human or animal (such as a fish), the process comprising:

i) providing a microbial cell of the invention described above,

ii) culturing the cell such as to product the composition of the invention,

iii) purifying the protein composition therefrom.

Optionally step i) is preceded by the steps described above i.e. preparation of the vector and/or introduction into a host cell.

Optionally step iii) may be followed by formulating the composition as a vaccine. Vaccines are discussed in more detail below.

It will be appreciated that while these steps produce very pure product, further preparative steps are not excluded. These may include gel filtration, PEG, ammonium sulfate or ethanol precipitation, acid extraction, phosphocellulose chromatography, hydrophobic interaction chromatography, affinity chromatography, hydroxylapatite chromatography, reverse phase chromatography, preparative electrophoresis, detergent solubilization, selective precipitation, centrifugation, ultracentrifugation, density gradient centrifugation, ultrafiltration through a size exclusion filter, and so on. General techniques are further described in, for example, R. Scopes, Peptide Purification: Principles and Practice, Springer-Verlag: N. Y. (1982).

Vaccines and Uses

The present invention provides vaccines comprising the composition of the invention or composition nucleic acids as defined above, and uses thereof as vaccines. In particular, the present invention envisages the use of the compositions of the invention in vaccines against disease in fish or other marine animal. In principle, the payload can comprise an antigen from any fish or other marine animal pathogen, meaning, the compositions described herein have the capacity to vaccinate fish or other marine animal against any pathogen.

A significant advantage of vaccines comprising the compositions of the present invention is that they can be administered without handling the fish or other marine animal (e.g. via immersion or oral routes; see example). Thus, the vaccines of the present invention present a low cost, low-labour and handling-stress free alternative to vaccination by injection, the current method of choice in the aquaculture industry.

Whilst it may be desirable to administer vaccines of the present invention without handling the fish or other marine animal, it is also possible to administer vaccines of the present invention by injection. The injected vaccines can comprise an adjuvant and be administered in an effective amount to a human or animal (such as a fish or other marine animal) in order to elicit an immune response. In preferred embodiments, the compositions are administered without an adjuvant to a human or animal (such as a fish or other marine animal). It is believed that such administration without an adjuvant will decrease the occurrence and/or severity of “local reactions”.

In one embodiment, the vaccine of the invention comprises a composition nucleic acid as defined above. In this embodiment, the vaccine can be administered by injection. The vaccine may be administered with or without an adjuvant.

Furthermore, vaccines can be produced in which the composition's payload contains antigens from multiple different fish (or other marine animal) pathogens, in principle allowing for simultaneous vaccination against multiple diseases. Alternatively, multiple simultaneous vaccination can be achieved by, for example, preparing an immersion solution containing several different (for example, 2, 3, 4, 5, 6, 7, 8, 9 or 10) different compositions or composition nucleic acids of the invention; immersion of a fish (or other marine animal) in this solution will result in vaccination against the antigens ion each of the payloads.

In one aspect, the invention provides a vaccine which comprises:
(i) a fragment of SpHtp1¹⁻¹⁹⁸ as shown in FIG. 1, which fragment comprises SpHtp1 ²⁴⁻⁶⁸;
(ii) a fragment of SpHtp1a¹⁻²²¹ as shown in FIG. 1, which fragment comprises SpHtp1a²⁴⁻⁶⁹; or
(iii) a polypeptide having at least 60, 70, 75, 80, 85, 90, 95, 96, 97, 98, 99 or 100% identity to (i) or (ii) which is able to translocate across cell membranes;

In combination with one or more of a pharmaceutically acceptable excipient, carrier, buffer, stabiliser (e.g. protease inhibitor) or adjuvant. In preferred embodiments, the vaccine does not include an adjuvant. In some embodiments the fragment is no more than 150 amino acids long, such as no more than 130, no more than 125, no more than 100 or no more than 75 amino acids. In some embodiments the fragment is no more than 60 amino acids. In some embodiments the fragment is no more than 50 amino acids.

Vaccines and their uses will now be described in more detail.

Vaccines will comprise a composition of the invention or a composition nucleic acid. A vaccine may comprise only one type of composition or composition nucleic acid, or may comprise more than one composition or composition nucleic acid, such as 2, 3, 4, 5, 6, 7, 8, 9, 10 or more than 10 different types of composition or composition nucleic acid. Vaccines may comprise, in addition to the above one or more compositions or composition nucleic acids, a pharmaceutically acceptable excipient, carrier, buffer, stabiliser (e.g. protease inhibitor) or other materials well known to those skilled in the art. Such materials should be non-toxic and should not interfere with the efficacy of the active ingredient (i.e. the composition of the invention). The precise nature of the carrier or other material may depend on the route of administration, e.g. oral or immersion routes.

In some embodiments the vaccines of the invention can comprise components that increase the efficacy of uptake of the composition or composition nucleic acid of the invention by the organism to be immunized, for example cofactors of a payload.

In one aspect the invention provides use of a vaccine of the invention in the manufacture of a medicament for inducing an immune response in a human or animal. In one aspect the invention provides a vaccine of the invention for inducing an immune response in a human or animal. In some embodiments the immune response is the generation of antibodies against one or more antigens comprised in the payload. In some embodiments the animal is a fish or other marine animal, such as salmonids, catfish, carps, sea bass, flat fish, and Tilapia's. In particular: Grass carp (Ctenopharyngodon idella), Silver carp (Hypophthalmichthys molitrix), Catla (Cyprinus catla or Gibelion catla), Common Carp (Cyprinus carpio), Bighead carp (Hypophthalmichthys nobilis or Aristichthys nobilis), Crucian carp (Carassius carassius), Oreochromis niloticus Nile Tilapia (Oreochromis niloticus), Mozambique Tilapia (Oreochromis mossambicus) and other Tilapia's, Pangas catfishes (Pangasius pangasius), Roho (Labeo rohita), Atlantic salmon (Salmo salar), Arctic charr (Salvelinus alpinus), brown trout (Salmo trutta), rainbow trout (Oncorhynchus mykiss), sea trout (Salmo trutta) and other trouts.

The invention also provides methods of reducing the likelihood of contracting a condition associated with infection by a pathogen in a human or animal (such as a fish or other marine animal), comprising administering an immunologically effective dose of a vaccine of the invention. For example, the method may reduce the likelihood of contracting a condition associated with infection by, i) bacterial pathogens, for example Vibrio anguillarum & Vibrio salmonicida (vibriosis), Aeromonas salmonicida & Aeromonas hydrophila (furunculosis), Yersinia ruckeri (Enteric Redmouth Disease), Renibacterium salmonis, Lactococcus sp., Streptococcus sp., and Piscirickettsia salmonis; (ii) viral pathogens, for example Infectious pancreatic necrosis virus (IPNV), Infectious Salmon Anemia virus (ISAV), Salmon Pancreas Disease virus (SPD virus), Sleeping Disease of Trout virus (SDV), Viral Nervous Necrosis virus (VNNV) and Infectious Heamatopoietic Necrosis virus (IHNV), Viral haemorrhagic septicaemia virus (VHSV); (iii) Fungal pathogens, for example Saprolegnia sp. (such as S. parasitica, S. diclina and S. australis) or Aphanomyces sp. (A. invadans and A. astaci) and Branchiomyces sp. (Gill rot); and/or (iv) Parasitic pathogens, for example Ichthyophthirius multifiliis, Cryptocaryon irritans and Trichodina sp. (Trichodiniasis), Amoebic gill disease (eg. Neoparamoeba perurans), sea lice (copepods within the order Siphonostomatoida, family Caligidae (for example Lepeophtheirus salmonis, Caligus rogercresseyi, Caligus clemensi, Caligus elongatus), proliferative kidney disease or PKD (Tetracapsuloides bryosalmonae) and other species)

Thus in some aspects of the present invention, the vaccines can be utilized in a vaccine strategy to induce an immune response in a human or animal. The vaccines can comprise an adjuvant and be administered in an effective amount to a human or animal (such as a fish or other marine animal) in order to elicit an immune response. In other preferred embodiments, the compositions are administered without an adjuvant to a human or animal (such as a fish or other marine animal).

The vaccines of the present invention can be administered using any technique currently utilized in the art. Suitable dosing regimens are preferably determined taking into account factors well known in the art including age, weight, sex and medical condition of the subject; the route of administration; the desired effect; and the particular composition employed. The vaccine can be used in multi-dose vaccination formats.

In one embodiment, a dose would consist of the range from about 1 μg to about 1.0 mg of the composition of the invention per subject kg. In another embodiment of the present invention the range is from about 0.01 mg to 1.0 mg of the composition of the invention per subject kg. However, one may prefer to adjust dosage based on the amount of protein delivered. In either embodiment, these ranges are guidelines. More precise dosages can be determined by assessing the immunogenicity of the composition of the invention so that an immunologically effective dose is delivered. An immunologically effective dose is one that stimulates the immune system of the patient to establish an immunological response. Preferably, the level of immune system stimulation will be sufficient to develop an immunological memory sufficient to provide long term protection against disease caused by the pathogenic organism from which the payload antigen(s) originates.

In some embodiments, the animal to be vaccinated is a fish and the composition of the invention is delivered by immersing the fish in an immersion solution comprising the composition of the invention.

Thus, the present invention provides a method for vaccinating a fish or other marine animal, the method comprising: (i) providing an immersion solution comprising a composition of the invention; (ii) immersing a fish or other marine animal to be vaccinated in the immersion solution; (iii) incubating the fish or other marine animal to be vaccinated in the immersion solution for a treatment period.

In some preferred embodiments of the above immersion vaccination methods, the composition of the invention is administered without an adjuvant.

In some embodiments, the animal to be vaccinated is a fish and the composition of the invention (or composition nucleic acid) is delivered by injection. In preferred embodiments, the compositions are administered without an adjuvant.

In the above immersion or injection vaccination methods, the composition of the invention will have a payload comprising an antigen from a fish or other marine animal pathogen. For example, an antigen from one or more of Vibrio anguillarum, Vibrio salmonicida, Aeromonas salmonicida, Aeromonas hydrophila, Yersinia ruckeri, Renibacterium salmonis, Lactococcus sp., Streptococcus sp., Piscirickettsia salmonis, Infectious pancreatic necrosis virus (IPNV), Infectious Salmon Anemia virus (ISAV), Salmon Pancreas Disease virus (SPDV), Sleeping Disease of Trout virus (SDV), Viral Nervous Necrosis virus(VNNV), Infectious Heamatopoietic Necrosis virus (IHNV), Saprolegnia sp. (such as S. parasitica, S. diclina and S. australis) or), Aphanomyces sp. (A. invadans and A. astaci)), Branchiomyces sp., Ichthyophthirius multifiliis, Cryptocaryon irritans and Trichodina sp., Amoebic gill disease (eg. Neoparamoeba perurans), sea lice (copepods within the order Siphonostomatoida, family Caligidae (for example Lepeophtheirus salmonis, Caligus rogercresseyi, Caligus clemensi, Caligus elongatus), proliferative kidney disease or PKD (Tetracapsuloides bryosalmonae).

In the above immersion vaccination methods, the concentration of the composition of the invention in the immersion solution will be sufficient to allow for vaccination of the immersed fish in a reasonable period of time. For example, the concentration of composition in the immersion solution can be from 0.1 to 10 μM, such as 1 to 5 μM, 2 to 4 μM or around 3 μM.

The treatment period can be, for example, 15 min to 3 hours, such as 30 mins to 2 hours or 1 to 1.5 hours. Other useful treatment period are, for example, 3 hours to 6 hours, such as 4 hours to 5 hours or at least 6 hours.

Accordingly, the present invention provides a method for vaccinating a fish or other marine animal, the method comprising: (i) providing an immersion solution comprising a composition of the invention at a concentration between 0.1 and 10 μM, wherein the composition of the invention has a payload comprising an antigen from a fish or other marine animal pathogen; (ii) immersing a fish or other marine animal to be vaccinated in the immersion solution; (iii) incubating the fish or other marine animal to be vaccinated in the immersion solution for a treatment period from 15 minutes to 3 hours. Preferably, the method is performed without the use of an adjuvant.

Preferably, an immune (e.g. antibody) response will be elicited in a fish or other marine animal subjected to the above immersion vaccination method. For example, at a timepoint 8 weeks after vaccination the fish or other marine animal may have a blood titer of at least 1:8, for example at least 1:16 or at least 1:32, such as at least 1:64, at least 1:128, at least 1:256, at least 1:512, or at least 1:1024.In some embodiments the fish or other marine animal has have a blood titer of at least 1:32 at a timepoint 8 weeks after vaccination. In some embodiments a titer of “at least 1:x” means the antibody is antibody is detectable against the antigen at that dilution via ELISA.

Thus, in one aspect the invention provides a method for manufacturing antibodies the method comprising the steps of: (i) vaccinating a fish (or other marine animal) with a composition or composition nucleic acid of the invention so as to elicit an antibody production; (ii) harvesting the antibodies from the fish (or other marine animal).

The present invention provides a composition of the invention for use in the above immersion or injection vaccination methods. In an embodiment, the invention provides a composition or composition nucleic acid of the invention for use in immersion or injection vaccination of a fish or other marine animal without the use of an adjuvant. The present invention also provides an immersion solution for use in the above immersion vaccination methods.

In an embodiment, the invention provides a composition of the invention for use in immersion vaccination of a fish or other marine animal without the use of an adjuvant, the composition having a payload comprising an antigen from a fish pathogen; wherein immersion of the fish or other marine animal for a treatment period from 15 minutes to 3 hours in an immersion solution containing the composition of the invention at a concentration between 0.1 and 10 μM elicits an immune (e.g antibody) response in the fish or other marine animal.

The present invention provides a composition of the invention for use in the immersion or injection vaccination of fish or other marine animal.

In some embodiments the composition of the invention is delivered by oral administration in food i.e. the composition of the invention is combined with food prior to administration. Thus, the present invention provides human or animal feed comprising: (i) a composition of the invention, and (ii) human or animal foodstuff. For example, in the vaccination of fish or other marine animal the animal feed of the invention would comprise the inventive composition together with typical fish or other marine animal food stuffs such as fish meal, fish oil, starch (wheat, maize gluten, sunflower meal). The present invention further comprises a method for vaccinating a human or animal (such as a fish or other marine animal) by feeding the human or animal the feed of the present invention.

The timing of doses depends upon factors well known in the art. After the initial administration one or more booster doses may subsequently be administered to maintain antibody titers. An example of a dosing regime would be a dose on day 1, a second dose at 1 or 2 months, a third dose at either 3, 4, 5, 6, 7, 8, 9, 10, 11, or 12 months or greater than 12 months, and additional booster doses at distant times as needed.

Decisions on dosage etc, is within the responsibility of medical practitioners, and typically takes account of the disorder to be treated or prevented, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

The immune response so generated can be completely or partially protective against disease and symptoms caused by infection with the pathogenic organism from which the payload antigen(s) originates.

### FIGURES

FIG. 1. Amino acid sequences of SpHtp1 fragments

FIG. 2.
A) Beakers with X-ray Tetra's swimming in a suspension of tank water and 3 μM SpHtp1-mRFP, or 3 μM mRFP, or no protein added (blanc). Fish remained in the suspension for one hour.
B) Finn, gill and bone tissue were crudely dissected and placed under the Confocal Microscope after one hour of immersion. The ‘no protein’ control is not shown, but there was no red-fluorescence observed in the fishes.

FIG. 3. ELISA results after immersion treatment of rainbow trout
C1.1 =Salmon treated with mRFP(His)₆ only
E1.1 =Salmon treated with SpHtp1²⁴⁻⁶⁸mRFP(His)₆
E2.1 =Salmon treated with SpHtp1²⁴⁻⁶⁸mRFP(His)₆

Graphs show the Vₘₐₓ rates of the detection at two different serum dilutions tested (“16” and “512”).

### EXAMPLES

### Example 1: ELISA results—Testing for Rainbow Trout IgG Capable to Bind mRFP(His)₆ After Immersion Vaccination (See Also FIG. 3)

Three salmon were exposed to protein constructs by immersion; two experimental animals (E1.1 & E2.1) were exposed to were exposed to SpHtp1²⁴⁻⁶⁸:mRFP(His)₆, whilst one control animal (C1.1) was exposed to mRFP(His)₆ only. Exposure was by 1.5 hour immersion in tank water comprising 3 μM of the relevant composition.

After 4 and 6 week blood samples were taken and tested by ELISA for binding to mRFP(His)₆: the results are shown in FIG. 3. As can be seen, both experimental fish test positive for antibodies against mRFP. Whilst lower than E1.1, the antibody levels observed in E2.1 are sufficient for antibody immunity. It is also noted that variation in immune response between individuals is not unexpected.

Note that it is likely a higher antibody titer would have been recorded at 8 weeks. However, the data at 8 weeks was not obtained as the experiment was prematurely terminated.

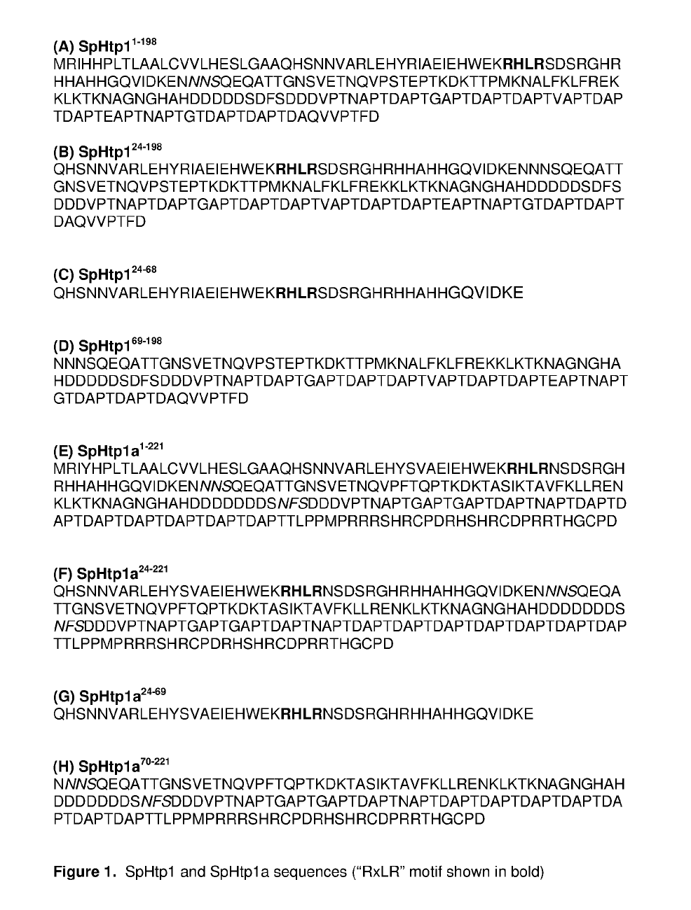

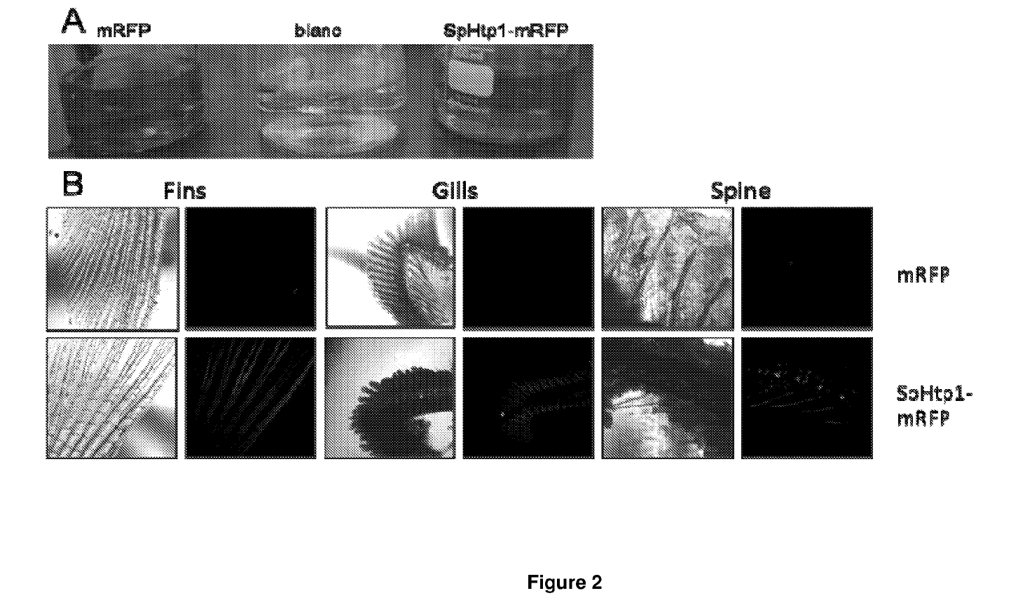

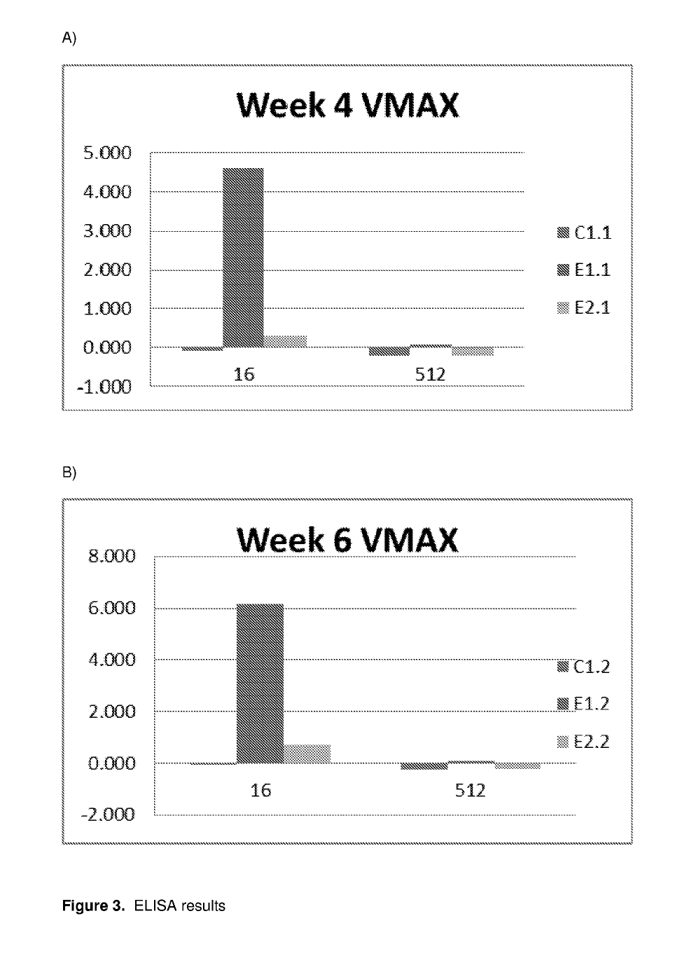

1. A composition comprising: (i) a translocation sequence comprising a polypeptide having at least 60% sequence identity to the SpHtp1²⁴⁻⁶⁸ or SpHtp1a^{2″9} shown in FIG. 1; and (ii) a heterologous payload coupled to the translocation sequence.

2. The composition of claim 1 wherein the composition comprising: (i) a translocation sequence consisting of a polypeptide having the sequence of SpHtp1²⁴⁻⁶⁸ or SpHtp1a²⁴⁻⁶⁹ shown in FIG. 1; and (ii) a heterologous payload coupled to the translocation sequence.

3. The composition of claim 1 wherein the heterologous payload is a peptide.

4. The composition of claim wherein the composition is a fusion protein.

5. The composition of claim 1 wherein the heterologous payload is a polypeptide from an organism or virus selected from Vibrio anguillaram, Vibrio salmonicida, Aeromonas salmonicida, Aeromonas hydrophlia, Yersinia ruckeri, Renibacterium salmonis, Lactococcus sp., Streptococcus sp., Piscirickettsia salmonis, Infectious pancreatic necrosis virus (IPNV), Infectious Salmon Anemia virus (ISAV), Salmon Pancreas Disease virus (SPD virus), Sleeping Disease of Trout virus (SDV), Viral Nervous Necrosis virus (VNNV) and Infectious Heamatopoietic Necrosis virus (IHNV), Viral haemorrhagic septicaemia virus (VHSV), S. parasitica, S. diclina. S. australis, A. invadans, astaci, Branchiomyces sp., Ichthyophthirius multifiliis, Cryptocaryon irritans, Trichodina sp., Neoparamoeba perurans, Lepeophtheirus salmonis, Caligus rogercressevn, Caligus clemensi, Caligus elongates or Tetracapsuloides bryosalmonae.

6. The composition according to claim 1 wherein the heterologous payload is a polypeptide, or polypeptide encoded by a sequence selected from the following, or a fragment thereof:
Genbank accession numbers ACY35988.1, ACY35989.1, ACY35990.1, EU625675.1, FJ594325.1, AJ012631.1, AJ238578.1, X89213.1, EU481506.1, ACA34520.1, ACA34521.1, ACA34522.1, ACA34523.1, ACA34524.1 or ACA34525.1;
UniProt accession numbers D0VF01, D0VF02, D0VF03, C6ETL2, C6G756, Q9WJ34, Q08449, 008454, 008453, 082706, 008445, Q08454, Q82707, Q8QL52, or Q08455.

7. A nucleic acid encoding a composition of claim 1.

8. An expression vector comprising the nucleic acid of claim 7.

9. A host cell comprising the expression vector of claim 8.

10. A vaccine comprising a composition according to claim 1, optionally in combination with a pharmaceutically acceptable excipient, carrier, buffer or stabilizer.

11. The vaccine of claim 10 for inducing an immune response in a human or animal.

12. The vaccine of claim 11, wherein the immune response is the generation of antibodies against one or more payload antigens.

13. (canceled)

14. A methods of reducing the likelihood of a human or animal contracting a condition associated with infection by a pathogen, the method comprising administering an immunologically effective dose of the vaccine of claim 10.

15. The method of claim 14, wherein the human or animal is a fish or other marine animal.

16. A method for vaccinating a fish or other marine animal, the method comprising:
(i) providing an immersion solution comprising a composition according to claim 1;
(ii) immersing the fish or other marine animal in the immersion solution;
(iii) incubating the fish or other marine animal in the immersion solution for a treatment period.

17. The method of claim 16, wherein the immersion solution contain the composition at a concentration of between 0.1 and 10 μM and the treatment period is between 15 minutes and 3 hours.

18. The method of claim 16 wherein the method is performed without the use of an adjuvant.

19. (canceled)

20. The vaccine of claim 1 wherein the human or animal s a fish or other marine animal.


SEQUENCE LISTING (33 sequences)

SEQ ID NO: 1 - protein, 198 aa, Saprolegnia parasitica
MRIHHPLTLAALCVVLHESLGAAQHSNNVARLEHYRIAEIEHWEKRHLRSDSRGHRHHAHHGQVIDKENNNSQEQATTGNSVETNQVPSTEPTKDKTTPMKNALFKLFREKKLKTKNAGNGHAHDDDDDSDFSDDDVPTNAPTDAPTGAPTDAPTDAPTVAPTDAPTDAPTEAPTNAPTGTDAPTDAPTDAQVVPTFD

SEQ ID NO: 2 - protein, 175 aa, Saprolegnia parasitica
QHSNNVARLEHYRIAEIEHWEKRHLRSDSRGHRHHAHHGQVIDKENNNSQEQATTGNSVETNQVPSTEPTKDKTTPMKNALFKLFREKKLKTKNAGNGHAHDDDDDSDFSDDDVPTNAPTDAPTGAPTDAPTDAPTVAPTDAPTDAPTEAPTNAPTGTDAPTDAPTDAQVVPTFD

SEQ ID NO: 3 - protein, 45 aa, Saprolegnia parasitica
QHSNNVARLEHYRIAEIEHWEKRHLRSDSRGHRHHAHHGQVIDKE

SEQ ID NO: 4 - protein, 130 aa, Saprolegnia parasitica
NNNSQEQATTGNSVETNQVPSTEPTKDKTTPMKNALFKLFREKKLKTKNAGNGHAHDDDDDSDFSDDDVPTNAPTDAPTGAPTDAPTDAPTVAPTDAPTDAPTEAPTNAPTGTDAPTDAPTDAQVVPTFD

SEQ ID NO: 5 - protein, 221 aa, Saprolegnia parasitica
MRIYHPLTLAALCVVLHESLGAAQHSNNVARLEHYSVAEIEHWEKRHLRNSDSRGHRHHAHHGQVIDKENNNSQEQATTGNSVETNQVPFTQPTKDKTASIKTAVFKLLRENKLKTKNAGNGHAHDDDDDDDSNFSDDDVPTNAPTGAPTGAPTDAPTNAPTDAPTDAPTDAPTDAPTDAPTDAPTDAPTTLPPMPRRRSHRCPDRHSHRCDPRRTHGCPD

SEQ ID NO: 6 - protein, 198 aa, Saprolegnia parasitica
QHSNNVARLEHYSVAEIEHWEKRHLRNSDSRGHRHHAHHGQVIDKENNNSQEQATTGNSVETNQVPFTQPTKDKTASIKTAVFKLLRENKLKTKNAGNGHAHDDDDDDDSNFSDDDVPTNAPTGAPTGAPTDAPTNAPTDAPTDAPTDAPTDAPTDAPTDAPTDAPTTLPPMPRRRSHRCPDRHSHRCDPRRTHGCPD

SEQ ID NO: 7 - protein, 46 aa, Saprolegnia parasitica
QHSNNVARLEHYSVAEIEHWEKRHLRNSDSRGHRHHAHHGQVIDKE

SEQ ID NO: 8 - protein, 152 aa, Saprolegnia parasitica
NNNSQEQATTGNSVETNQVPFTQPTKDKTASIKTAVFKLLRENKLKTKNAGNGHAHDDDDDDDSNFSDDDVPTNAPTGAPTGAPTDAPTNAPTDAPTDAPTDAPTDAPTDAPTDAPTDAPTTLPPMPRRRSHRCPDRHSHRCDPRRTHGCPD

SEQ ID NO: 9 - protein, 260 aa, Infectious pancreatic necrosis virus
MLPENGPASIPDDITERHILKQETSSYNLEVSDSGSGLLVCFPGAPGSRVGAHYRWNLNQTELEFDRWLETSQDLKKAFNYGRLISRKYDIQSSTLPAGLYALNGTLNAATFEGSLSEVESLTYNSLMSLTTNPQDKVNNQLVTKGITVLNLPTGFDKPYVRLEDETPQGSQSMNGARMRCTAAIAPRRYEIDLPSERLPTVAATGTPTTIYEGNADIVNSTTVTGDVTFQLAAEPANETRFDFILQFLGLDNDVPVVSV

SEQ ID NO: 10 - protein, 260 aa, Infectious pancreatic necrosis virus
MLPETGPASIPDDITERHILKQETSSYNLEVSESGSGLLVCFPGAPGSRIGAHYRWNANQTGLEFDQWLETSQDLKKAFNYGRLISRKYDIQSSTLPAGLYALNGTLNAATFEGSLSEVESLTYNSLMSLTTNPQDKVNNQLVTKGVTVLNLPTGFDKPYVRLEDETPQGLQSMNGAKMRCTAATAPRRYEIDLPSQRLPPVPATGTLTTLYEGNADIVNSTTVTGDINFSLAEQPAVETKFDFQLDFMGLDNHVPVVTV

SEQ ID NO: 11 - protein, 260 aa, Infectious pancreatic necrosis virus
MLPETGPASIPDDITERHILKQETSSYNLEVSESGSGILVCFPGAPGSRIGAHYRWNANQTGLEFDQWLETSQDLKKAFNYGRLISRKYDIQSSTLPAGLYALNGTLNAATFEGSLSEVESLTYNSLMSLTTNPQDKVNNQLVTKGVTVLNLPTGFDKPYVRLEDETPQGLQSMNGAKMRCTAAIAPRRYEIDLPSQRLPPVPATGALTTLYEGNADIVNSTTVTGDINFSLAEQPAVETKFDFQLDFMGLDNDVPVVTV

SEQ ID NO: 12 - DNA, 1122 bp, Infectious salmon anemia virus
atggcacgattcataattttattcctactgttggcgcctgtttacagtcgtctatgtcttagaaactaccctgacaccacctggttaggtgactctcgaagcgatcagtccagagtgaatccacagtctttggatctggtgactgagttcaagggggtgctgcaggccaaaaacggaaatggacttttgaagcagatgagtggaaggtttccaagtgactggtacacacctactacaaagtaccggatcctatacttgggaaccaatgactgcactgacggacctactgacatgatcatcccaacttcgatgacactggacaacgcggcaagggagctgtacctgggagcatgcaggggagacgtgagagtgacgcctacatttgtgggagcagcaattgttggacttgttggacgaacagacgcaattaccggtttttcggtgaaggtgttgactttcaacagccctacaattgtagtggtgggattgaatggaatgtccggaatctacaaggtctgcattgcagcaacatctgggaatgtgggaggagtgacacttatcaacggatgcggatatttcaacacacctttgaggtttgacaatttccaaggacaaatctacgtgtcagacacctttgaagtgaggggaaccaaaaacaagtgtgttctgctaagatcttctagtgatacgcctttgtgttcacacatcatgaggaacgttgagttagatgagtatgtagacacaccaaatacagggggtgtttatccttctgatggttttgattcactacatggttcagcttccgttagaacgttcctcactgatgcattgacatgcccagacattgactggagtagaattgatgctgcttcgtgtgaatatgacagctgccctaagatggttaaagattttgaccagacaagcttaggtaacacagacacacttatcatgagggaggtagcattgcacaaggagatgatcagtaaacttcagaggaacatcacagatgtaaaaacatctgtgttgagcaacatcttcatttctatgggtgtagcaggttttgggattgctctgttcctagcaggttggaaggcatgtatttggattgcagcattcatgtac

SEQ ID NO: 13 - DNA, 1186 bp, Infectious salmon anemia virus
atggcacgattcataattttattcctactgttggcgcctgtttacagtcgtctatgtcttagaaactaccctgacaccacctggttaggtgactctcgaagcgatcagtccagagtgaatccacagtctttggatctggtgactgagttcaagggggtgctgcaggccaaaaacggaaatggacttttgaagcagatgagtggaaggtttccaagtgactggtacacacctactacaaagtaccggatcctatacttgggaaccaatgactgcactgacggacctactgacatgatcatcccaactccgatgacactggacaacgcggcaagggagctgtacctgggagcatgcaggggagacgtgagagtgacgcctacatttgtgggagcagcaattgttggacttgttggacgaacagacgcaattaccggtttttcggtgaaggtgttgactttcaacagctctacaattgtagtggtgggattgaatggaatgtccggaatctacaaggtctgcattgcagcaacatctgggaatgtgggaggagtgacacttatcaacggatgcggatatttcaacacacctttgaggtttgacaatttccaaggacaaatctacgtgtcagacacctttgaagtgaggggaaccaaaaacaagtgtgttctgctaagatcttctagtgatacgcctttgtgttcacacatcatgaggaacgttgagttagatgagtatgtagacacaccaaatacagggggtgtttatccttctgatggttttgattcactacatggttcagcttccgttagaacgttcctcactgatgcattgacatgcccagacattgactggagtagaattgatgctgcttcgtgtgaatatgacagctgccctaagatggttaaagattttgaccagacaagcttaggtaacacagacacacttatcatgagggaggtagcattgcacaaggagatgatcagtaaacttcagaggaacatcacagatgtaaaaacatctgtgttgagcaacatcttcatttctatgggtgtagcaggttttgggattgctctgttcctagcaggttggaaggcatgtatttggattgcagcattcatgtacaagtctagaggtagaattccaccatcgaacctgtttgttgcttgattatgaaagaaagacaacc

SEQ ID NO: 14 - protein, 374 aa, Infectious salmon anemia virus
MARFIILFLLLAPVYSRLCLRNYPDTTWLGDSRSDQSRVNPQSLDLVTEFKGVLQAKNGNGLLKQMSGRFPSDWYTPTTKYRILYLGTNDCTDGPTDMIIPTSMTLDNAARELYLGACRGDVRVTPTFVGAAIVGLVGRTDAITGFSVKVLTFNSPTIVVVGLNGMSGIYKVCIAATSGNVGGVTLINGCGYFNTPLRFDNFQGQIYVSDTFEVRGTKNKCVLLRSSSDTPLCSHIMRNVELDEYVDTPNTGGVYPSDGFDSLHGSASVRTFLTDALTCPDIDWSRIDAASCEYDSCPKMVKDFDQTSLGNTDTLIMREVALHKEMISKLQRNITDVKTSVLSNIFISMGVAGFGIALFLAGWKACIWIAAFMY

SEQ ID NO: 15 - protein, 388 aa, Infectious salmon anemia virus
MARFIILFLLLAPVYSRLCLRNYPDTTWLGDSRSDQSRVNPQSLDLVTEFKGVLQAKNGNGLLKQMSGRFPSDWYTPTTKYRILYLGTNDCTDGPTDMIIPTPMTLDNAARELYLGACRGDVRVTPTFVGAAIVGLVGRTDAITGFSVKVLTFNSSTIVVVGLNGMSGIYKVCIAATSGNVGGVTLINGCGYFNTPLRFDNFQGQIYVSDTFEVRGTKNKCVLLRSSSDTPLCSHIMRNVELDEYVDTPNTGGVYPSDGFDSLHGSASVRTFLTDALTCPDIDWSRIDAASCEYDSCPKMVKDFDQTSLGNTDTLIMREVALHKEMISKLQRNITDVKTSVLSNIFISMGVAGFGIALFLAGWKACIWIAAFMYKSRGRIPPSNLFVA

SEQ ID NO: 16 - DNA, 5178 bp, Salmon Pancreas Disease virus
actatggactcagcggcaatgaacgtggaggcttttaaaagtttcgcctgtaaggacaccgacctgtggactgagttcgcggaaaaaccagtaaggttgtcgcccggccaaatcgaagagtatgtctttcatctacaaggggccaaggccaatgtgatgcacagcagagtcgaagccgtatgccctgacctctcggaggtggctatggacaggttcacactagacatgaaacgcgacgtcaaagtgacgccaggcacgaagcacgtagaggagagacctaaagtccaagagattcaagcggccgaccccatggccaccgcgtacttgtgcgccatccatagagagctagtccgaaggctgaaggccgtcctgaaaccgtctatacacgtgttgttcgatatgagctccgaggattttgatgctatcgtgggccatgggatgaagttgggtgacaaggtgctggaaacggacatctcctcattcgacaagagccaggaccaagccatggcggttacagcgctgatgctgctgagggacttgggagtagaagaagacctcctgaccctaattgaggcgtctttcggcgacatcacttctgcccacctgcccacaggcaccagatttcagtttggatcgatgatgaagtctggactttttctgacgctgttcgtgaacacgctgcttaacatcaccatagctgcccgagttttacgggagcagctggctgataccaggtgtgccgcgtttatcggtgacgacaacgtaatcaccggagtagtgtctgacgacatgatggtggccaggtgcgcatcctggctgaacatggaggtgaagatcatggacatggaaattggcaacatgagtccttatttttgtggcggcttcctgttactcgacacggtaacaggcactgtaagccgagtgtcggaccctgtaaaacgcctgatgaagatgggaaaaccggccctgaacgatccagaaacggacgtggacagatgccgcgcactgcgcgaagaagtggaaagctggtacagagtggggattcagtggccactgcaggtggctgccgccacacgctatggcgtgaaccacctgccgctggccacaatggcgatggccacgctcgcccaggacttgagatcgtacctgggcgcgcgaggggagtacgtatccctctacgtctaaccttaatattttctgcatcatacttccaaacaatcatgtttcccatgcaattcaccaactcagcctatcgccagatggagcccatgtttgcaccgggttcccgaggacaagtacagccgtaccggccgcgcactaagcgccgccaggagccgcaagtcggcaacgccgccattactgccctcgcgaaccagatgagtgcgctccagttgcaggtagctggacttgccggccaggcaagggtggaccgccgtgggccaagacgtgttcagaagaacaagcagaagaagaagaactcttccaacggagaaaaacccaaagagaagaagaagaagcaaaaacaacaggagaagaagggaagcggtggcgaaaaagtcaagaagactaggaaccgacccgggaaggaggtaaggatctccgtaaagtgtgcccgacagagcaccttccccgtgtaccacgaaggtgctatatccggctacgctgtgctgattggatctcgcgtattcaagccggcacacgtgaagggtaagatcgaccaccctgaactggcagacatcaagttccaggtcgccgaggacatggacctcgaagcagctgcgtacccgaagagcatgcgagaccaagcggctgaaccagcgaccatgatggacagagtgtacaactgggagtatggcactatcagagtggaggataatgtcataatcgacgcaagcggtaggggcaagccgggtgacagtggcagggccatcaccgacaactcgggaaaggttgttggtattgtcctcggaggaggacccgatggcaggcgcacacgcctctccgtgataggtttcgacaagaagatgaaggctagggagatcgcctacagtgatgccataccttggacacgcgctccggccctcctgctgctgcctatggttattgtctgcacctacaattccaacaccttcgattgctccaaaccgtcctgccaggactgctgcattactgctgaaccagagaaggccatgaccatgctgaaggacaatctgaacgacccgaactactgggacctactcattgctgtcaccacctgtggctccgcccggagaaagagggctgtgtctacgtcgcctgccgccttttacgacacacagatcctcgccgcccacgcagctgcctccccatacagggcgtactgccccgattgtgacggaacagcgtgtatctcgccgatagccatcgacgaggtggtgagcagtggcagcgaccacgtcctccgcatgcgggttggttctcaatcgggagtgaccgctaagggtggtgcggcgggtgagacctctctgcgatacctgggaagggacgggaaggttcacgccgcagacaacacgcgactcgtggtgcgcacgactgcaaagtgcgacgtgctgcaggccactggccactacatcctggccaactgcccagtggggcagagcctaaccgttgcggccacactggatggcacccggcatcaatgcaccacggttttcgaacaccaagtaacggagaagttcaccagagaacgcagcaagggccaccatctgtccgacatgaccaagaaatgcaccagattttccactacaccaaaaaagtccgccctctacctcgttgatgtgtatgacgctctgccgatttctgtagagattagcaccgtcgtaacatgcagcgacagccagtgcacagtgagggtgccacctggtaccacagtgaaattcgacaagaaatgcaagagcgctgactcggcaaccgtcactttcaccagcgactcccagacgtttacgtgtgaggagccagtcctaacggctgccagtatcacccagggcaagccacacctcagatcggcaatgttgcctagcggaggcaaggaagtgaaagcaaggatcccgttcccgttcccgccggaaaccgcaacttgcagagtgagtgtagccccactgccgtcgatcacctacgaggaaagcgatgtcctgctagccggtaccgcaaaataccctgtgctgctaaccacacggaaccttggtttccatagcaacgccacatccgaatggatccagggcaagtacctgcgccgcatcccggtcacgcctcaagggatcgagctaacatggggaaacaacgcgccgatgcacttttggtcatccgtcaggtacgcatccggggacgctgatgcgtacccctgggaacttctggtgtaccacaccaagcaccatccagagtacgcgtgggcgtttgtaggagttgcatgcggcctgctggctatcgcagcgtgcatgtttgcgtgcgcatgcagcagggtgcggtactctctggtcgccaacacgttcaactcgaacccaccaccattgaccgcactgactgcagcactgtgttgcataccaggggctcgcgcggaccaaccctacttggacatcattgcctactttttaggggtaagagggtggtcagccctgctggtcatccttgcgtatgtacagagctgcaagagctacgaacacaccgtggtggtcccaatggatccaagagccccgtcgtacgaagcagtgataaaccggaatgggtatgatccattgaagctgaccatctcagtgaatttcaccgtcatctcaccaactacggctctggaatattggacctgcgcaggagtccccatcgtcgagccgccccatgtgggctgctgcacgtcggtgtcctgcccctctgacctctctacgctgcatgcgtttactggcaaagctgtctccgacgtgcactgcgatgtgcacacaaacgtgtaccccttgttgtggggcgcggctcactgcttctgttccaccgagaatacacaggtcagcgctgtggcagccaccgtttctgagttctgtgcccaggactcagagcgtgccgaagcgttcagcgtacacagcagctcagtcaccgctgaggtcctggtgacgcttggtgaagtggtgacggcagtccacgtttacgtggacggggtaacatcagccaggggcactgacctcaagatcgtggctggaccaataacaaccgactactccccattcgatcgcaaagtagtccgcatcggcgaagaggtctataactatgactggcctccttacggggctggccgaccaggcacattcggagacattcaagctaggtcaaccaactatgtcaaacccaacgatctgtatggggacatcggaattgaagtactgcagccgactaacgaccacgtacatgtggcttacacgtatacgacctctgggttactgcgttggctgcaggacgctccgaaaccactcagtgtcacagcaccgcacggttgtaagatcagtgccaatccgctcctggccctcgattgtggggttggtgccgtccccatgtccatcaacattccggacgcgaagtttacccgcaaattaaaggatccgaaaccatcggccctgaaatgcgtggtggacagctgcgagtacggggtggactacgggggcgccgccacgatcacctacgagggccacgaggccgggaagtgcgggattcattccctgacaccaggagtccccctgagaacatcggtggttgaagtggttgctggcgccaataccgtcaaaacgaccttctcctcacccacgcccgaggttgcactcgaggtagagatctgttcggcaatagtgaagtgcgctggtgagtgcactccaccgaaggaacatgtggtcgcaaccaggcctcgccatggcagcgaccctggaggctacatctccgggcccgcaatgcgctgggccggagggattgtagggaccctagtggtcctgttccttatccttgccgtcatctactgcgtggtgaagaagtgccgctccaaaagaatccggatagtcaagagctaaattccggtatacaaattgctcactaggagcccatccgatcccacagggagtaggatgagtcatctattggttttaaaattttcaatacaaaaaaaaaaaaaaa

SEQ ID NO: 17 - protein, 394 aa, Salmon pancreas disease virus
TMDSAAMNVEAFKSFACKDTDLWTEFAEKPVRLSPGQIEEYVFHLQGAKANVMHSRVEAVCPDLSEVAMDRFTLDMKRDVKVTPGTKHVEERPKVQEIQAADPMATAYLCAIHRELVRRLKAVLKPSIHVLFDMSSEDFDAIVGHGMKLGDKVLETDISSFDKSQDQAMAVTALMLLRDLGVEEDLLTLIEASFGDITSAHLPTGTRFQFGSMMKSGLFLTLFVNTLLNITIAARVLREQLADTRCAAFIGDDNVITGVVSDDMMVARCASWLNMEVKIMDMEIGNMSPYFCGGFLLLDTVTGTVSRVSDPVKRLMKMGKPALNDPETDVDRCRALREEVESWYRVGIQWPLQVAAATRYGVNHLPLATMAMATLAQDLRSYLGARGEYVSLYV

SEQ ID NO: 18 - DNA, 4179 bp, Sleeping disease virus
cacaatggcgatggccacgctcgcccaggatctgagatcgtacttgggcgcgcgaggggagtacgtatccctctacgcctaaccttaatattttctgcatcatactctcaaccaaccatgtttcccatgcaattcaccaactcagcctatcgccagatggagcccatgttcgcaccggcttctcgaggacaagtacagccgtatcggccgcgcacaaagcgccgccaagagccgcaagtcggcaacgctgctattgctgccctcgcgaaccagatgagcgcgctccagctgcaggtggctggacttgccggccaggcaaggttggaccgtcctggaccgagactttttccagaaaaaacaagcagaagaagaagaactcttccaacggagaaaaacccccggagaagaagaaagaagccaaaaccacctggagaagaaagggaacggcggggaaaaagccaagaaacccccggaaccggccccggaaagaagttaggatctccgttaagcgtgcccgacagaacaccttccccgttgtaccatgacggtgccatatccggctatgcggtgctgattggctcccgcgtgtttaagccagcgcacgtgaagggtaagttcgaccaccccgaactggcggacatcaagttccaggtcgccgaggtcatggacctcgaagcagccgcataccctaagtgcatgcgagaccaggcggctgaaccagcaaccatgatggatggagtgtacaatggggagtacggcaatattcaggagtggaggacaattttgtattcgatgcgagcggcagaggcaagccggggtgacagtggcaggccattcaccgacaactcaggaaaggttgtcggtatcgtcctcggaggaggacccgatggtaggcgcacacgtctctccgtgataggtttcgacaagaagctgaaggccagagagatcgcctacagcgaggccatcccttggacacgcgcaccagctctcctgctgctgcctatggtcatcgcctgcacctacaactccaatacctttgattgctccaaaccgtcctgccaggattgttgcattactgctgaaccaaagaaggccatgactatgctgaaggacaacctgaatgacccgaactactgggacctgctcattgccgtcaccacctgcagttccgcccgaaaaaagagggctgtgtctacgtcgcctgtcgccgtttacgacacacaaattctcgccgcccacgcagctgcctccccgtatagggcgtactgccccgattgtgacggaactgcctgcatctcgccgatagctatcgacgaggtggtaagtagcggtagtgaccacgtccttcgcatccgggtcggttctcaatcgggagtgaccgctaaaggcggtgcggcgggtgaaacctctctgcgatacctgggaagggacggtaaggtttacgccgcggacaacacgcggctcgtggtgcgcaccactgcaaagtgtgacgtgctgcaggccactggccactacattctggccaactgcccagtggggcagagtctcactgttgcggccacactggacggtacccggcatcaatgcaccacggttttcgaacatcaagtaacggagaagttcacaagagaacgcagcaagggccaccacctgtccgatctgaccaagaaatgcaccaggttctccaccaccccgaagaagtccgcgctctatctcgttgatgtgtatgatgctctgccgacttctgtagagatcagcaccgtggtgacatgcaacgaaagacagtgcacagtgagggtgccacccggtaccacagtgaaattcgataagaggtgcaagaacgctgccaaagagaccgtcaccttcaccagcgactcccagacgtttacgtgcgaggagccggtcctaacggccgccagcatcacccagggcaagccgcacctcagatcgtcaatgttgcccagcggaggcaaagaggtgaaagcgaggattccattcccgttcccgccagagactgcgacttgcagagtgagcatcgccccactgccatcgattacctatgaggaaagcgatgttctgctggccggcactgcgaaataccccgtgctgctaactacacggaaccttggtttccatagcaacgccacatctgaatggatccagggtaagtacctgcgccgcatcccggtcacgccccaagggattgaactaatgttgggaaacaacgcaccgctgcacttctggtcatctgtcaggtacgcatctggagacgccgacgcgtacccctgggaacttctggtgcaccacatcaagcaccatccggagtacgcgtgggcgtttgtaggagttgcatgtggcctgctggccgttgcagcatgcatgttcgcgtgcgcatgcaacagggtgcggtactctctgctcgccaacacgttcaacccgaacccaccaccattgaccgcactgactgcagcattgtgctgcatacctggggctcgcgcggatcaaccctacctggacatcattgcctacttgtggaccaacagcaaagtggccttcgggctgcaatgcgcggcgcccgtggcttgcatgctcatcgttacatacgcccttagacattgcagattgtgctgcaattcttttttaggggtaagagggtggtcggctctgctggtcatccttgcgtatgtacagagctgcaaggcgtacgaacacaccgtggtggtcccaatggatccaagagccccgtcgtacgaggcggtgataaaccggaatgggtatgaccccctgaagcttaccatcgcagtgaactttaccgtcatctcaccaactacggctctggaatactggacctgtgcaggagtccctgtcgtcgagccgccccatgtgggctgctgcacgtcagtgtcctgcccctccgacctctccacgctgcacgcgttcaccggcaaagccgtctccgacgtgcactgcgatgtgcacacgaacgtgtaccccttgttgtggggtgcggctcactgcttctgttccactgaaaacacgcaggtcagcgctgtggccgccaccgtttctgagttctgtgctcaggactcagagcgcgccgaggcgttcagcgttcacagcagctcagtcactgcagagattctggtgacgcttggtgaagtggtgacggcggtccacgtttacgtggacggggtaacatcagccaggggtaccgacctcaagatcgtggctggcccaataacaactgactactccccgtttgaccgcaaagtagtccgtatcggcgaagaggtctataattacgactggcctccttacggggctggtcgaccaggcacattcggagacattcaagctaggtcaaccaactatgtcaaacccaatgatctgtacggggacatcggaattgaagtactgcagccgactaatgaccacgtgcacgtggcttacacgtatacgacctctgggttgctgcgttggttgcaggacgctccgaaaccactcagtgtcacagcaccgcacggttgtaagatcagtgctaacccgctcctggccctcgattgtggggttggtgccgtccccatgtccatcaacattccggacgcgaagttcacccgcaaactaaaggacccgaaaccttcggccctgaaatgcgtggtggacagttgcgagtacggggtggactacgggggcgccgccacgatcacctacgagggccacgaggctgggaagtgcgggatccattccctgacaccaggagtccctctgagaacatcagtggttgaagtagttgccggcgctaataccgtcaaaacgaccttctcctcacccacgcccgaggttacactcgaggtagagatctgttcggcaatagtgaagtgcgccagtgagtgcactccaccgaaggaacacgtagtcgcagccaggcctcgccatggcagcgacactggaggctacatctccgggcccgcaatgcgctgggccggaaggattgtagggaaccctagtggtcctgtttcctcatccttggccgtcacctactgcgtggtgaagaagtgccgctctaaaagaatccggatagtcaagagctaaattccggtatacaaatcgctcactaggagcccatccgaacccacagggagtaggatgagtcatctattggtttttaaaattttcaat

SEQ ID NO: 19 - protein, 1322 aa, Sleeping disease virus
MFPMQFTNSAYRQMEPMFAPASRGQVQPYRPRTKRRQEPQVGNAAIAALANQMSALQLQVAGLAGQARVDRRGPRRVQKNKQKKKNSSNGEKPKEKKKKQKQQEKKGSGGEKAKKPRNRPGKEVRISVKRARQSTFPVYHDGAISGYAVLIGSRVFKPAHVKGKFDHPELADIKFQVAEVMDLEAAAYPKCMRDQAAEPATMMDGVYNGEYGNIQEWRTILYSMRAAEASRGDSGRPFTDNSGKVVGIVLGGGPDGRRTRLSVIGFDKKLKAREIAYSEAIPWTRAPALLLLPMVIACTYNSNTFDCSKPSCQDCCITAEPKKAMTMLKDNLNDPNYWDLLIAVTTCSSARKKRAVSTSPVAVYDTQILAAHAAASPYRAYCPDCDGTACISPIAIDEVVSSGSDHVLRIRVGSQSGVTAKGGAAGETSLRYLGRDGKVYAADNTRLVVRTTAKCDVLQATGHYILANCPVGQSLTVAATLDGTRHQCTTVFEHQVTEKFTRERSKGHHLSDLTKKCTRFSTTPKKSALYLVDVYDALPTSVEISTVVTCNERQCTVRVPPGTTVKFDKRCKNAAKETVTFTSDSQTFTCEEPVLTAASITQGKPHLRSSMLPSGGKEVKARIPFPFPPETATCRVSIAPLPSITYEESDVLLAGTAKYPVLLTTRNLGFHSNATSEWIQGKYLRRIPVTPQGIELMLGNNAPLHFWSSVRYASGDADAYPWELLVHHIKHHPEYAWAFVGVACGLLAVAACMFACACNRVRYSLLANTFNPNPPPLTALTAALCCIPGARADQPYLDIIAYLWTNSKVAFGLQCAAPVACMLIVTYALRHCRLCCNSFLGVRGWSALLVILAYVQSCKAYEHTVVVPMDPRAPSYEAVINRNGYDPLKLTIAVNFTVISPTTALEYWTCAGVPVVEPPHVGCCTSVSCPSDLSTLHAFTGKAVSDVHCDVHTNVYPLLWGAAHCFCSTENTQVSAVAATVSEFCAQDSERAEAFSVHSSSVTAEILVTLGEVVTAVHVYVDGVTSARGTDLKIVAGPITTDYSPFDRKVVRIGEEVYNYDWPPYGAGRPGTFGDIQARSTNYVKPNDLYGDIGIEVLQPTNDHVHVAYTYTTSGLLRWLQDAPKPLSVTAPHGCKISANPLLALDCGVGAVPMSINIPDAKFTRKLKDPKPSALKCVVDSCEYGVDYGGAATITYEGHEAGKCGIHSLTPGVPLRTSVVEVVAGANTVKTTFSSPTPEVTLEVEICSAIVKCASECTPPKEHVVAARPRHGSDTGGYISGPAMRWAGRIVGNPSGPVSSSLAVTYCVVKKCRSKRIRIVKS

SEQ ID NO: 20 - DNA, 11137 bp, Infectious haematopoietic necrosis virus
gtataaaaaaagtaacttgactaagctcagaaggacacaaggacagaaaaaagaaaaaggtggcacttcagttgtaacctgataacgtgcttcagacactataaaccgagacagaacaagcagaactattttcactgaaaacaacacctgagagacagaaacggatcacgaacgatgacaagcgcactcagagagacgttcactggactcagagacatcaaggggggagtcctcgaagatgcagagacggagtatcgtcccggtacgataaccctccctctatttttctccaagacagactttgacctagagatgatcaagcgggcggtgagtcaagtcggaggagagggaacgagaagggcattgggcctcctgtgcgcgttcgtcattgcagagacggtcccatcggaggcaggtacggtcgccgaacttctggaagccctgggctttgtgctggagtctttggacactggggcaccaccagacgctaccttcgcagaccccaacaacaagcttgcagaaacgatcgtaaaggaaaatgtccttgaggttgtgaccggcctcctcttcacctgcgccctactgacaaagtatgatgtggacaagatggccacatactgccaaaacaagctcgagcgtcttgcaaccagccaagggattggcgagttggttaacttcaacgccaacaggggagtcctggccaggatcgggtcggtgcttagacccggacagaagctcaccaaggctatctatgggatcattctcatcaacctgtccgacccagccatcgctgccagagccaaggcactgtgcgccatgagactgagcgggacaggaatgacaatggtgggactgttcaaccaagccgcaaagaacctgggcgcccttccagccgaccttctagaggatctgtgcatgaagtcagtggtggagtccgccagacgcattgtcagactgatgaggatcgtagcagaggccccaggggtagcagcaaagtacggtatcatgatgagcaggatgctcggggaggggtacttcaaggcctacgggatcaacgagaacgccaggatcacctgcattctcatgaacatcaacgataggtatgacgatgggacctcgagaggactgacagggataaaggtatccgaccctttcaggaagctggcgagagagatcgctcgtctccttgtcctcaagtacgatggcgatggctcaaccggagagggggcgtccgagctgatccgccgggcggagatggcatctcggggaccagacatgggtgaggaagaggaggaggacgaggaggacgactactccagtgagccaggagactccgattcattccgctgaacacaaacagcccccccctcctttttcctctcccgcccctcgacccatccagatgtttactccaccctgactcttagatagaaaaaaatggcactatagtgcttttcaactcaaaccacaacaatgtcagatggagaaggagaacagttcttcaacctagaaggcgaagacatactgaggctagaatcccgcctgaaaaacccacggaatgacgggcaaatcggcaagaaccccaggcaacggaaggaggaccaggcgcctcaagaggaaccaaagaaaaccaccaggagacccgacaagaacaagggtctatctcaactggagcaactcatcctaaagtacgttgaggaggagagctgtcaggatgccctgaaggacttcggaggtctaattgccaacatcagacaggcccaccaggccgaactgacatctcacctagaaaaggttgctacggaacaccgagccaatcttcaggctcttacaaagtcccagcaagagcacgagaaagtctcgaaggagattttgtctgcggtaattgctattcggtccaacctcaacgagaaccacagtcccaggcccaagccactcgacccggatcaggtgaaggccgctcgtgcccttggattcggaattgggtatcgaacggcccttaatgtcttcgaccgaataaagggagtcaccccagacaacgcaggatcccaagaggtgaagaacatggccattcgggcggcggaggaggatgaatacgagggaagtcccactttcttcaggaaggtgatagacgccgtaaagaagcgcatgaagcaaggtcaatagacatcctcctccaggatcccggctaccaagacagaaaaaaatggcacgcaagtgtatcgttccaaacgaaatccaagagtcagttcaaacgagagcatgtctattttcaagagagcaaagagaacagttctgatccctcctcctcacctcctcagcggagacgaggagagggttacaatactcagtgcagagggggagatcaaggtgactggaagaagaccaaccacacttgaggagaagatctattactctatgaatctggcggctgccatcgtagggggagatctacacccctcattcaaatccatgacctatctgttccagaaagagatggagttcggaagcacccaagaaaaggtcaacttcggctctcggaaaccagcacctcagaccacctaccaggtaatgaaggcgagggaaatctaccttcagacccagcctctcgagaagaagatcccaatgcagacctactcagtcagcacagagggggctaccatcaccttcacaggacggttcctgttttcatccagccatgtaggttgcgacgacaacaggaccaaactggcggggcttgacgggttcacaacctccaacagctaccagagggtcaaagactactatgcccaggagacagccctggccctgaccttcgcggctcccgaaaagcgggggaaggaaaaataggagtcgaccatgccgtctctcactcacccatccatcggccgacccaaccctcctccatccccagagtccctccactcctcactccgtccaagacagaaaaaaatggcacttttgtgctttgagacagaacgcaactcgcagagacccaccaaaacaatggacaccacgatcaccactccgctcattctcattctgatcacctgcggagcaaacagccaaaccgtcaaacccgacaccgcaagcgaatcagaccaacccacctggtcaaacccgctcttcacctatcccgagggatgcactctggacaagctctccaaggtcaatgcctctcagctgagatgcccaaggatcttcgacgatgagaacagggggctaattgcctatcccacatccatccggtccctgtcagtcggaaacgacctcggggatattcacacccaagggaaccacatccacaaagtcctgtaccgcaccatctgctcaacagggttcttcgggggtcagacgatagagaaggcgcttgtagaaatgaaactctctacgaaagaagcaggggcgtatgacaccacaaccgcagccgctctgtacttcccagctccccgatgccaatggtacaccgacaacgtacaaaacgatctcatcttctactacacaacccaaaagagtgttctgagagatccctacaccagagactttctggactcagattttattggaggaaaatgcaccaaatcaccctgccagactcattggtccaacgtcgtttggatgggtgatgcagggataccagcctgtgattccagccaagagataaaaggtcacctctttgttgataaaatctccaatcgagtcgtgaaggcaacgagctacggacaccacccctggggactgcatcaggcctgtatgattgaattctgtgggaaacagtggatacggacagatctcggtgacctaatatctgtcgaatacaattctggagcagaaatcctctcgttcccgaagtgtgaggacaagactgtggggatgaggggaaacttggatgactttgcctatctagacgatcttgtgaaggcctctgagagcagagaggaatgtcttgaggcgcacgccgagataatatcaacaaacagtgtgactccatacctcctatccaagttccgatctccacatcccggaataaatgacgtctacgctatgcacaaaggctccatctatcacgggatgtgcatgacggtcgctgtggacgaggtatccaaggacaggacaacttacagggcccatcgcgctaccagtttcacgaaatgggaacgaccctttggggatgagtgggagggctttcacggattgcacggaaacaacaccaccattattccagacctggagaaatacgtcgcccagtacaagacgagcatgatggaaccgatgagcatcaaatcagtaccccatccaagcatcctggccctctacaatgagacagacgtatcagggatctccatcaggaaattggactcattcgaccttcaatcactccactggagtttctggcccacaatctccgcactgggtgggattccctttgctctcctccttgctgttgccgcgtgctgctgctggtcagggagacctcccactccctccgcgtcgcagagtatccccatgtatcacctggcaaaccggtcctaaaggacctcaatcctcacttcctccccaccagacagaaaaaaacggcacatctgtgctgcaaaaagagacaatggaccaccgcgacataaacacgaacatggaggcactcagagaagttctgcgatacaagaacaaggtggccggacacggcttcctctttgacgacggagacctggtatggcgtgaagaggacgacgcaacgtggaggcggctttgcgatgtcgtcaacgcactgatctcctccaagaggatgcagcgagtattgtacatggacctcagcatcaccaagggcgaggggcatctactttttgtggatctccaggggaccaagaaccgcctgcacaaagaaccccgattcaggagacatctgatcctgattgaagactttcttgcttatcccagatagaaaaaaatggcacttttgtgcaaaaaaactcaagcgcgactcacagagaacacaaccagcaacgacagcaacccatcacagaaaaatggacttcttcgatcttgacatagaaataaaacaagagagacttccagcggaatgctccctcaactcacccctaaattactcactgtctgcacaactgacagatcgcatgacccctagaactgagaacgtcaggaggcaaagagaaaggctcaggtcccacatgcgagaacacttccgggtcaaggatctatccaccctcgacaatgactccaccaggctccatgcacgactcacagaggatctcatcaccattcaaagccccgagatcgattcctcagtccttgaaaattggaccccacttaagtcctactatgcatctctagactacacactcccggaaaagacagccttcaagtgggaacacgcagccccctactggaatctatttgcacaactgagggcaattctcctccagagccaaaagatcaagaaacaagagacgggcacgagggaactctactcttgtggccctcttcagatagagttcgtggagggggtagtcttgtacttcacagatcgaggatcgaaagaggaattcaccaagtcaggagagctcccaagtgtaacaccgtatgcggacttccttgcctggatcaagatcatctcccaacgggcccaggccgtgttgatggcagtcatcctcagagtgaccgacaagggactatctccactaccagaatcactcctggccatctatcaaaacgtggacgacatcctgaaacgagcgggccagcctgcgatagacctcctgaaactctgggagccattggtgattacaaaattgggagaacttctcggagacagattcggactagaagaagatttccgaaacacaatacgaggtgaggcaacaaaattggcaaagaacctattcatttcacgcgggctcaatcgcctcatggacatcctggatcagcaaactgacgctcaaccgctctttcaatttttcggactattcaaacattttgcgtacccacgtgtcttctcaagagacaccatccaggcaatccaggaggtaagtgacaggcctagctcaatctcagctgtcgactttctccatgatcagtgcgagattcgcaaggaattctacgtcagatacataaacgcataccacagggcacctggccttgacctctccgccctgagtccctcctcatttctaagagacagtctcgagcgtggaaaaatcccaaacgaaagaagccctctctattcaaacaaagagtggtacttcgtcaaattcacaaaaagcatcgagtggccagttagcgacactctgtcgacattcctatcggacaaagcgatcactcgtgaccgacccgcctggattgaggacgggcactcgggtagagacatgtccgagaagcggctgctactcaaatttatcaaggagaacttctcgagtgttgcagacattgtcggtgcggcggaagcgatttacaacaaccaagaggaccgactgatcgcactgaaagtgaaagagatggaactaaagatcaagggcaggggattcgggctgatgaccttcatgcctcgtctcctccaagtcctccgtgagagcatcgccaaaaaaacacaaaaactattccctgagatcaccatgacctcctcagatctggacatgaaaaaaagaaagttcatgctctcaaaaaagagcgacgacaggcgaggcttcatacacgtgaataaaagtctggacatcaacaaattttgcaccagccagagacaattcaactccaatgcggttttctcaagtctagatgaactcatggggacctttcctttgttttcccgagttcacgaaatttttgaaaaaacctggatcgtagacgggtcatcctcggatccccccaacttggcacacttcactagaattctcgacgaatgcacggcacttggtctagacacccctcacatctgggcggacggggtcttctcggggttgaagggagggatcgaagggctctgtcaatacgtttggactatctgtcttttacttagagtagaacgcgtcatgcaaaaaacagcgctcacccactacattctggcacagggcgacaatgtgattatcacaattatcgtaccagtagaaatacatcgggatggcaccattccagaacaagagtcccgtcgtatcttggccttaagtcgagacatcgatctctcactggagagtgaactagaaaaaagtggtctcaccctaaagatcgaagagactctaacaagtgagaacatctccatctacgggaaggatcttcactgtccgcaacaccttaccctggcaatcaagaaggccgcctccgccgcgatcatatcaagtgagcagtaccaagatgtcccaacatttctatctggtctcggaacgagtctggaggccctctcagagtgtgtcaacaacaaagtcggggtgcatctctttggagtgatcatgggcgttgcgggatggagggacctggcgactcatcagacttggcgaggatggagatacccctatcacaagaaggcgataactgggcggattcgggcatccgagatgaagctcagcaaaggcgaacccaccgagctgtccatctctgtcctctccaagcgacgtagagagaccgaaacactcattgaactactatccaacagcctgctcgggtcagccctaggaatgcttgcgttccctactcctctagacctggaaaaacggggtgtgggagactacatcacccatcggctcaccatagcacgcaaggcccttctgagtggacaccttgatccccgtatcgggagaaaagttgaatcagcatgcaacatccccctctcgtccagaaccgacctaagcaaactgtttgactctccattttcattgaacatcgcaaccgaagaggacgcgacggcagtcatcaaacgacaggcaacaaaaatcctccgactgcaagagataaaaaacgagaagctgaaggcccagattgacaacatggacaagggcatagcgacccttgatgcagcacttgcaggagccaccaacatcaaccccagactgaattacatgatcaggagcatcacggacgaaaaggagtccgagatgtttgtcactaaattcgcatcagcaagaaccatgaggaccctggcaatgaatcactcctcagaacttcccattgtcatcctcctggagatgaaaagccaacaaaaagagacatacacaatctggagaaccaagagaccacctgtgaccatgtggaaatgctccacagtgttggcgaaggagctccgagacacctcctggggcaaaaacataatcggaggcacatccccttctccaatcgaggcaatggagacaatccaaattgaccccaccgaatgggaagaccgacggtcccaggacgccatgtccatcaactactatctgtcacgggctggcatggacgaacagacagcaaagctcactcgagggttcctagtcccttactacgggacccaaacaaaaccccttgtggccaaggcctacctagaactcaagggcaacccccgaaccaacaaggctctccttctcctgagtgttcgagagtctctcgtaaaaacaggcagcaatctcgacaagttgatcataaagctctgctcccatgcactagacatcgatgtagcctcactccccgcccttcgagcacaggaagaggcggcagctggggagggtctccgaggaggaatcaaagaatccatgtctccagtaggtcccgataacttctacacaaacatcacgcacaaggtcttcaacaggaagtgggcaacaccataccacgtcaacattgcagacttcatcatccaaggcctgatagagaccagaaggcacctccttgtaaatgagaggatgaatgggctactcccggtcagctcagtcaaatgcacctcctgcttcaggaagaaagagagggagttctttgatattcccgaggaattcacctggaaaaatgagtcaaaaacctcagatcccgcctacacctacttcaccacctggtgtgacctccccagggtctccaacctgcccgagatggatcaacggtcagccactcgtctcttgggaagagggctcgcattaaacagatcctcctccggtgaaatcatcaccaaattctactcaatgcccatggaatcacaacggctccttcaccccgtggaactgctcctgggatatggtgaaggggttatatttggatatttgaggagtcagcacataaatcacggagccctctttcacattggcgacgagtcactggcaaagaaactcagacgttatgtcctagacacaaagacacaacatgccaagcaaattggctacctcttccaggatgaggactcattacatgagctcctgggccaaggactttgtccatatatacccaggtcaatccctcttaccatcactgaactaacaaacgcctgcgcgatcacaaccatcagggcaacggaggtaatcctaagcacaaaagccaggatccaccatatgcccgtccaggcaatcgacgaatcagatgtcgacaccagcagactagctgcgaacaccatgcagaccatactcggagaccccagacccatgaacctcgtgcatcttgactgcgatctcacccacaacatggtcgcatgggagtcagaagtggaactcgacatcttgaaatcagaaaacttccacattgacggactcctggtagaactcactgcccgcgagctccccattggagacaccccctggaagcaaagagactggacctgttccaacgatcccaagatcatcgccaagggcatcaagacaaaatccctgttcattcatcagggcgtgactggggcaatcaacttgattcccgatcttctggtcgtgatcgggggaggtctcggagggtgcgccgttccctaccttcaggaatggccagacacaccaatcatctttgcaaccctattcgacgaacgggagaggatctccgaggatggagacttgattgttcccccagagctcctggtgaggggcatggcacctaggatgatcgagagggagatcttagaagcagagctgtgtgacatcaccaatgaggggaatcgtcgcctgctaattcgactggtcacgaaaaacaagggaggaggcaaggtggtgctcatagacgagatcgagaatcgaggggccccagaatcccttctacagagctcgctccaagacctattcgagaaactggacaaagtctgcaaactcacctctgtgcacacagtcagagagtcaacagtggaacaattctcccagagggtaaattctatcaaacgcagcaggaaggcagtaacactgcattggaaccgctacaacagaagagaccaatttgaggccctggtcatcgtgaagggcgaagaggccaagtccgactaccggatctccacgatcacctcggcaaaggcatttcgcaagatcgacgaacagctcgagatagacgggagactctcgtcgacacagtggtccctccccgcgcttccttctcgagaaaaaaacattctttttgggtacgtgtcatccgtcttccttaagatgaaccttgcactctcagccaacgacatggaccgagagcgactgatcgagaccattgagggaaccgccccagggttgatctcctggaaggaaaaactcgaacacagggaccatgcacatcgctccgacattgaagagaaaggaataactcaggacaagatcttcaacctcatctgcctctcctgggtcctcaagggtctgagatacggagtatgggacaccgacgctcaatccattgtagctcaaacggtgtacatcactcgagggccaaagctctgtccattgggggagaagcccaagcgcatcttcgcatcattcaaactacaaagcggaaagcgagttgaagatgccaagggattcctgagcgcactgcttcaccttgagggcttctttccactaggcgaacaataggtacactccagcagcacccagtgccagggaccaaactcccagatacaaaaaaatggctcccgactcccatggtaagggaaccagtcattgttttctgttattaaaaaccttttttaactgccttttgagaacccttctgttactttttttat

SEQ ID NO: 21 - protein, 391 aa, Infectious hematopoietic necrosis virus
MTSALRETFTGLRDIKGGVLEDAETEYRPGTITLPLFFSKTDFDLEMIKRAVSQVGGEGTRRALGLLCAFVIAETVPSEAGTVAELLEALGFVLESLDTGAPPDATFADPNNKLAETIVKENVLEVVTGLLFTCALLTKYDVDKMATYCQNKLERLATSQGIGELVNFNANRGVLARIGSVLRPGQKLTKAIYGIILINLSDPAIAARAKALCAMRLSGTGMTMVGLFNQAAKNLGALPADLLEDLCMKSVVESARRIVRLMRIVAEAPGVAAKYGIMMSRMLGEGYFKAYGINENARITCILMNINDRYDDGTSRGLTGIKVSDPFRKLAREIARLLVLKYDGDGSTGEGASELIRRAEMASRGPDMGEEEEEDEEDDYSSEPGDSDSFR

SEQ ID NO: 22 - protein, 111 aa, Infectious hematopoietic necrosis virus
MDHRDINTNMEALREVLRYKNKVAGHGFLFDDGDLVWREEDDATWRRLCDVVNALISSKRMQRVLYMDLSITKGEGHLLFVDLQGTKNRLHKEPRFRRHLILIEDFLAYPR

SEQ ID NO: 23 - protein, 230 aa, Infectious hematopoietic necrosis virus
MSDGEGEQFFNLEGEDILRLESRLKNPRNDGQIGKNPRQRKEDQAPQEEPKKTTRRPDKNKGLSQLEQLILKYVEEESCQDALKDFGGLIANIRQAHQAELTSHLEKVATEHRANLQALTKSQQEHEKVSKEILSAVIAIRSNLNENHSPRPKPLDPDQVKAARALGFGIGYRTALNVFDRIKGVTPDNAGSQEVKNMAIRAAEEDEYEGSPTFFRKVIDAVKKRMKQGQ

SEQ ID NO: 24 - protein, 195 aa, Infectious hematopoietic necrosis virus
MSIFKRAKRTVLIPPPHLLSGDEERVTILSAEGEIKVTGRRPTTLEEKIYYSMNLAAAIVGGDLHPSFKSMTYLFQKEMEFGSTQEKVNFGSRKPAPQTTYQVMKAREIYLQTQPLEKKIPMQTYSVSTEGATITFTGRFLFSSSHVGCDDNRTKLAGLDGFTTSNSYQRVKDYYAQETALALTFAAPEKRGKEK

SEQ ID NO: 25 - protein, 508 aa, Infectious hematopoietic necrosis virus
MDTTITTPLILILITCGANSQTVKPDTASESDQPTWSNPLFTYPEGCTLDKLSKVNASQLRCPRIFDDENRGLIAYPTSIRSLSVGNDLGDIHTQGNHIHKVLYRTICSTGFFGGQTIEKALVEMKLSTKEAGAYDTTTAAALYFPAPRCQWYTDNVQNDLIFYYTTQKSVLRDPYTRDFLDSDFIGGKCTKSPCQTHWSNVVWMGDAGIPACDSSQEIKGHLFVDKISNRVVKATSYGHHPWGLHQACMIEFCGKQWIRTDLGDLISVEYNSGAEILSFPKCEDKTVGMRGNLDDFAYLDDLVKASESREECLEAHAEIISTNSVTPYLLSKFRSPHPGINDVYAMHKGSIYHGMCMTVAVDEVSKDRTTYRAHRATSFTKWERPFGDEWEGFHGLHGNNTTIIPDLEKYVAQYKTSMMEPMSIKSVPHPSILALYNETDVSGISIRKLDSFDLQSLHWSFWPTISALGGIPFALLLAVAACCCWSGRPPTPSASQSIPMYHLANRS

SEQ ID NO: 26 - protein, 1986 aa, Infectious hematopoietic necrosis virus
MDFFDLDIEIKQERLPAECSLNSPLNYSLSAQLTDRMTPRTENVRRQRERLRSHMREHFRVKDLSTLDNDSTRLHARLTEDLITIQSPEIDSSVLENWTPLKSYYASLDYTLPEKTAFKWEHAAPYWNLFAQLRAILLQSQKIKKQETGTRELYSCGPLQIEFVEGVVLYFTDRGSKEEFTKSGELPSVTPYADFLAWIKIISQRAQAVLMAVILRVTDKGLSPLPESLLAIYQNVDDILKRAGQPAIDLLKLWEPLVITKLGELLGDRFGLEEDFRNTIRGEATKLAKNLFISRGLNRLMDILDQQTDAQPLFQFFGLFKHFAYPRVFSRDTIQAIQEVSDRPSSISAVDFLHDQCEIRKEFYVRYINAYHRAPGLDLSALSPSSFLRDSLERGKIPNERSPLYSNKEWYFVKFTKSIEWPVSDTLSTFLSDKAITRDRPAWIEDGHSGRDMSEKRLLLKFIKENFSSVADIVGAAEAIYNNQEDRLIALKVKEMELKIKGRGFGLMTFMPRLLQVLRESIAKKTQKLFPEITMTSSDLDMKKRKFMLSKKSDDRRGFIHVNKSLDINKFCTSQRQFNSNAVFSSLDELMGTFPLFSRVHEIFEKTWIVDGSSSDPPNLAHFTRILDECTALGLDTPHIWADGVFSGLKGGIEGLCQYVWTICLLLRVERVMQKTALTHYILAQGDNVIITIIVPVEIHRDGTIPEQESRRILALSRDIDLSLESELEKSGLTLKIEETLTSENISIYGKDLHCPQHLTLAIKKAASAAIISSEQYQDVPTFLSGLGTSLEALSECVNNKVGVHLFGVIMGVAGWRDLATHQTWRGWRYPYHKKAITGRIRASEMKLSKGEPTELSISVLSKRRRETETLIELLSNSLLGSALGMLAFPTPLDLEKRGVGDYITHRLTIARKALLSGHLDPRIGRKVESACNIPLSSRTDLSKLFDSPFSLNIATEEDATAVIKRQATKILRLQEIKNEKLKAQIDNMDKGIATLDAALAGATNINPRLNYMIRSITDEKESEMFVTKFASARTMRTLAMNHSSELPIVILLEMKSQQKETYTIWRTKRPPVTMWKCSTVLAKELRDTSWGKNIIGGTSPSPIEAMETIQIDPTEWEDRRSQDAMSINYYLSRAGMDEQTAKLTRGFLVPYYGTQTKPLVAKAYLELKGNPRTNKALLLLSVRESLVKTGSNLDKLIIKLCSHALDIDVASLPALRAQEEAAAGEGLRGGIKESMSPVGPDNFYTNITHKVFNRKWATPYHVNIADFIIQGLIETRRHLLVNERMNGLLPVSSVKCTSCFRKKEREFFDIPEEFTWKNESKTSDPAYTYFTTWCDLPRVSNLPEMDQRSATRLLGRGLALNRSSSGEIITKFYSMPMESQRLLHPVELLLGYGEGVIFGYLRSQHINHGALFHIGDESLAKKLRRYVLDTKTQHAKQIGYLFQDEDSLHELLGQGLCPYIPRSIPLTITELTNACAITTIRATEVILSTKARIHHMPVQAIDESDVDTSRLAANTMQTILGDPRPMNLVHLDCDLTHNMVAWESEVELDILKSENFHIDGLLVELTARELPIGDTPWKQRDWTCSNDPKIIAKGIKTKSLFIHQGVTGAINLIPDLLVVIGGGLGGCAVPYLQEWPDTPIIFATLFDERERISEDGDLIVPPELLVRGMAPRMIEREILEAELCDITNEGNRRLLIRLVTKNKGGGKVVLIDEIENRGAPESLLQSSLQDLFEKLDKVCKLTSVHTVRESTVEQFSQRVNSIKRSRKAVTLHWNRYNRRDQFEALVIVKGEEAKSDYRISTITSAKAFRKIDEQLEIDGRLSSTQWSLPALPSREKNILFGYVSSVFLKMNLALSANDMDRERLIETIEGTAPGLISWKEKLEHRDHAHRSDIEEKGITQDKIFNLICLSWVLKGLRYGVWDTDAQSIVAQTVYITRGPKLCPLGEKPKRIFASFKLQSGKRVEDAKGFLSALLHLEGFFPLGEQ

SEQ ID NO: 27 - DNA, 11065 bp, Viral hemorrhagic septicemia virus
aataacggcacataagtggcaaaaagttttcaagtttgaaattccaagaatttggaaagtgaaagttgaacacagagtcatatctcgtaatctttgaacaaaagaactcagttgaagaatggacggaggacttcgtgcagcgttctcaggtttgaatgacgtcaggatcgaccccaccggtggagagggacgggtacttgtacctggtgaggtggagctcattgtgtatgtgggtgggtttagtgcagatgatgggaaggtgatcgtagatgcactttctgcacttgggggaccccagactgtgcaggcattgtccgttcttctctcctatgtacttcaagggaacaaacaggaggacctggaggcaaagtgcaaggtgctcacagacatgggcttcagggtgacacagtcaaccagggcgacgggcatcgatgcaggaatcctgatgcctatgagagagctggccctggccgtcaataacgacagcctcatggacatcgttaaggggaccctgatgacatgttcccttctgaccaagtactcggtggacaagatgatcaagtacatcaccaagaagcttggggacctggcagacactcagggaattggggaactgcagcacttcaccgccgacaaggcagccatcaggaaactcgcagggtgtgtgcgtcccgggcagaagatcaccaaggccctctatgcattcatcctgaccgagatcgcagatcccaccacccaatcaagggcccgagccatgggggcattgaggctcaacgggacaggaatgaccatgatcgggttgttcacccaggccgccaacaacctgggcattgccccggcgaagctgctggaggacctctgcatggagtctctggttgagtcagccagacggatcatccagctgatgagacaggtgtcagaggcaaagtccatccaagagcgctacgccatcatgatgagtcggatgctgggggaatcctactacaagtcgtacggactaaatgacaactccaagatctcctacattctgtcgcagatcagtgggaagtacgcagtggactccctggaaggcctggaggggatcaaggtgacagagaagttccgcgagttcgctgaacttgttgccgaggtcctggtggacaagtacgagaagattggggaggacagtactgaggtttcagatgtcatcagggaggcggctagacagcacgcgcgcaggacatccgccaagccagagcccaaggcccgcaacttcaggagccccaccggaagagggaaggagcaggatacagaggagtctgacgatgaggactaccccgaagactccgattaaaaagcactctcgtctcatgaccaacatagatagaaaaaaatggcacgattataagaatttctctttgatcaaagcaaggcaaacaccgaaatcacacaatggctgatattgagatgagtgaatccttggtcctgtcccatgggtccctagctgacctggataggaaactagacaacgcccccaaggacactaggtcagctcttttttcatctacctcagggttcaccaaacaaaaatcaagtcccaagaagaagcccaatcctacaacgctggaagaactcattggacacttcgtctctgaggatctccagctagacgccacaaaggccttcggacagctcctaagacgtatcaagatgtcccatcaggaagaacttactcaacatctggagaaggtcaacggagagaaccgggccaagatgggagccctcctggagtctcagaaagaaaatgggaagaagaccgacaacatactctcaatcctcatctccatgcgaggagaaggggcggaaaatgcatccaaaaagcccaaggtcctagatggggatcaggtccggaacgagagggcacttggattcaacaggggactgactacggctgccatcgccatgaagaagttcaagttagaagatcctctcgcactctgcaaaggctcagtcaaacgagcagctctctccgccatggaaaaggaggaatacgacggggagcgagagacctactccacagtctcaaaagcgattaaggcggagctggacaagctagagtagggacaacacaatgcatcgtacagatagaaaaaaacggcacgactgttcgagcaaacttactcccaaaaagagacactccagcagccagcaaaccccagacaatccacaactcaagacatgactctgttcaaaagaaagcgcaccatcctggttccccctcctcacatcacgtccaacgacgaggaccgtgtctcaaccatcctgacggaaggcactctaaccatcacgggtcctcctccgggaaaccaggtcgacaagatctgcatggctatgaagctggctcgagccattctctgcgaggatcagcacccagcattcaatcccctggtttatctattccagagtgctatgatctttggagagaccagtgagaagattgacttcgggactcgtagcaagaccctgatcacaagcttcaagatagctgaggcaaaggccatctacctggaatcaagtccggtgagatctcgcatagaagccaagaagtataccactcccatcagacatgggagtgtgacttactatggcccgttcgtgtttgcggatgaccacgttggagggaaaggccaccgcgagaaactgggggcactatgtgggtttctccagtcgggaccttacgggcaggcgaaggactactacaatcgtgctgtcgaggaagaaatgggcattctcccgagggacccaaagcgcagatctggagcctcttctgctcagccccggtaggtccaaatgacaaactcaggggtgataccatactcccattagatagaaaaaaatggcacatttgtgtacacaacaagctatacctacaatggaatggaatacttttttcttggtgatcttgatcattctcatcaagagcaccacatcacagatcacccaaagacctccgatcgagaacatctcgacgtaccatgcagattgggacactccgttgtacactcacccctccaactgcagagaagactcctttgtcccgattcgaccagctcaactcaggtgtcctcatgaatttgaagacatcaacaagggactggtctccgttccaactcagatcattcatctcccgctatcagtcaccagcgtctccgcagttgcaagtggccactacctgcacagagtgacctatcgagtcacctgctcaaccagcttctttgggggacaaaccatcgaaaagaccatcctggaagcaaaattgtctcgtcaggaggccgcaaatgaggcaagcaaagaccatgagtacccgttctttcctgaaccctcctgcatctggatgaaaaataatgtccataaggatataactcactattataagaccccaaaaacagtctctgtggatctctacagcaggaaatttcttaaccctgatttcatagagggggtttgtacgacctcgccctgtcagactcattggcaaggagtctattgggtcggtgccacacccacagcccattgtcccacttcagaaacactagaggggcacctgttcaccagaacccatgatcacagagtggtcaaggcaattgtggcgggccatcacccatggggactcacgatggcatgcacagtgacattctgtgggacagattggatcaagaccgacctgggagacctgatcaaggtggtaggacaggggggcgaaacaaaactgactccaaaaaaatgtgtcaatgctgacatccagatgaggggggcaacagacgacttctcatatctcaaccatctcatcacaaacatggctcaaaggaccgagtgtctagacgcccatagtgacatcactgcctccggtaaaatatcctcgtttctcctctcaaagtttcgtcccagccaccccggacccggcaaggcacattacctccttgatggtctaatcatgcgaggtgactgtgactatgaggcggtagtcagtatcaactacaatagcgctcagtacaagacggtgaacaacacatggaagtcatggaaacggatagacaacaatacagatgggtacgatgggatgatatttggggacaaactgatcatcccagacatcgagaaatatcagagtatctatgatagtgggatgctcgttcaaaggaacctggtggaagttcctcatctgagcattgtgtttgtctccaacacatctgatctctccaccaatcacatccacaccaacttaatcccctcggattggtcattcaactggagcctctggccgtcattatcagggatgggagttgtgggaggggccttccttctattggtactttgctgttgttgcaaagcgtctcctcctattccaaattacgggattcctatgcagcaattctccagaaatcagatggtctgaacacacctgcccgaatgaacatcattatcctcataggtagatagaaaaaaatggcacttctttgaaaaagaaacatgacaacccaactcgcgctcgacacagtcagcttttctccactcgtcctccgcgagatgatcacacacaggctcacattcgacccaagtaactacctcaactgcgacatcgatcggtcggacgtatccaccatgagtttcttcgaaacgactcttcccaggatcttggatgacttgagggcttgcccacggcttccctacctccatgtactcgacatgaggataagtctcctggagagaacccattacatgttcaaaaacgtcccctccagtcccgccacaaccggcagaccgactgatcctggactcatcattatttcatgtgcagatgttggggtcctaacaaatggctccgggttcacttcatgagcctcccccccgcccctcagatagaaaaaaatggcacttttgttgtttgtaatcctacgctcaacaaagaagtgaccctacagggggtaatacctgacacaatcaaattagatcaccttatagcaaccatggaaatgtttgaactagaccgggaagtacatcaggagcgacttccctccgaatgttcgctgaactctcccctaaacctttccctatctctccaactcttcgggcgcctaactccaaagacagaacacatcaggtaccaagctgggaggataaagagatggctcgtcaaacagtaccaactggtgcacctcagagagcttgagattgattcaacaagaatccaaggatatctgatcccccatcttttgaagacacagagtaatgagttaggatcgtcggtaataaaaaactgggggatggtctccaagtactacctgtctctagggtacacccttcccccaaaggacaagtttgactttcgagaagttgctccttactggaacttggcctcacagttgagagaggtcactttggagagccagaaggtggataccagagggaaagaaaaaagaaagttgtaccaggtggaagatgtagagttcgagtttaaagagggggtggtggtaattcgtgcgggcctggatggactcctgaatgagttccttgggggggaaaaacttggggcagtaacatatgtggaatacttggccttcttcaaaatcatcaatcaaagagcacaagcactcctgctcacggccatctgccaggcccttgaaccagacctagttcctccttgtagtggaatactttccgtctatgcagaggtcgactccgtactccgtcgggcgggacaatccgccattgacctgctcaaattgtgggaacctcttgtcctgacaaaactgggggacgtgctcggtgacagattcggcctcgaggacgacttcaaggacacaattaggggagaggcgaacaaattagctaagaaactccacgtcaccagatcttataaacggatgatgaagaccctagatcaggagactagggcacaggccctctttcaatattttggactgttcaaacacttcgcctacccccgcgtctattcgagagaaaccattgaggctattcaagaggtcagtgacaaaccgagtgactcgtccccactcaattatctaagtgaccaatgcaaaatcagggaggagttctacatccggtacacgaaggcttatcacagggcaccggccatgaacctgggtcaactaggacagggttcctacctacgccaggttttggaggcgggaaagatccccaacgccaagaatgccctctattcgctcctcgagtggttctttgttcgttttgagaagagcatcgaatggcccctcagtgacaccttatccaccttcctctcagacaaggcgatcacccaaaatagggacatctggtatgacgggggatcatcaggaagggacaccacagagaagagactactcctgaagtttatcaaggagaatgaagacagtgtggaaaaggtgatcctcaaggccgatgaaatctacgataaggaggcggatcagatcattgcactgaaagtcaaggagatggaactgaaaatcaaagggagaggatttggcttgatggctttcaagccaagactacttcaggtcctccgagagagtatcgccaagaagaccagcaagttattcccagaaataaccatgaccttttcagacctcgagttgaaaaagaaaaaatttcaactttctaggaagagcgatgacaggaggggatacatccatatcagcaaaagtcttgacataaacaagttctgcaccagtcaacgccagttcaactccctggcagtcttccagagcctagatgaacttctggggacagatcagctcttcacgagggtacacgagatcttcgaaaaaacatggatcgtggatggatccgccagtgaccccccagacctggtgacgttcaaggcgaggtatgaagaggcactggccctagggattgaggccccgcacgtgtgggcagacggagccttctctggactaatgggtggaattgagggtctctgtcagtacgtctggacaatctgtctcttactcagagtggagcgcgtcctcgctgtgacccaactgactcactttgtcatggcacaaggggacaatgtgatcatcaatctcatcatccctgttgaggtggaccgggtgggtggggttgtcgaaggggaacgtgccagaattcaacgcatcagcaaagacatcgactctgctctcgaacgagagctgctgaggagtggcttgactcttaagatagaggagaccctgaccagcgaaaacctctcaatctacggaaaagacctccattgcccacaacacctgacccttgccgtgaagaaggcaggatctgcctctatcatatcaagcgaacaatatcaagacgtgcccacctttctgtcaggcctcgggacgggaatggaaacaatctcagagtgcgtcaacaacaaagtgagtgctcatctgttcggggtcattctgggagcggctggatggaagagcctcgcacagagacaaacctggaaagggtgggaatacccctttcagaatgagacctcccggagacaggtccggtcacagggaatccttcttcagaagggggagtccacaatggtacacaaagagcccgaggccaacccggaaaaacggaccatagaactgctcctagtgagtagtcttttcggatcagctctggggatgctcccctttcccactcccattgaccttgagaagaggggcgtgggagactacgtcactcaccggctctcaatcgttaagatggccctggtctcaaaaaaactccccaataggatgatcgagatgattgtctccaccatgaaccttcccctttctcgagaacaggatctcactaagttattcgactctccattctccctcaatcttgccacagaggaagacgctgcatcagtgatcaagcgactcgcaaggggcacactaagggggcttgacatcaaaaacaaaaaactggcagatcacatagccaccatggatcagggaataacccaaattgacgaagcactggcaagtgcggacacaatcaaccccaggatcgcctaccaatttagaaacatcacagaccaaaaagagtcagaaatgttcgtcaccaagttcgccaccgcgaaaaccatgcggatggttgccctctcctcatctcaggatgtctcagttgtaggacttttgaacaaacgcagccaggcaaaagagatctacaccatttggcggacacagaggaagggggagacactctggacatgctcgacccagcaggcaaagaaactcagggaccgatcctggggaaaaaacataattggggtcacctctccttcccctctggaagccattctcttcaagctgattgatccaatttcctgggaggaagaaaaggaggcacaccacttcaccatccattactacctctcaaagcccagtctttcttcaaagacagcccacacgaccaggggtccacttgttccctacttcggaacccagaccaagccactgatcgcaaaagcctacatggagctcaagggaaatcccaggaccaacaaggctctgcagttgcttagcatgagggagaccatgatcaaggcagggtccaatctagacaaactcctcttgtccctgtgttctaacgccctagacatcgacgtcaactccctcccttcacttcaggcacaagaagaggccagtgctggagagggggtccgaggaggtatcaaagagtccatgtccccagttggaccggacaacctctacacacacatcacccacaaggtgttcgagcgtcagtggttgtctgagtttcatgtgaacatcgctgacttcatcatctggggaatcacgaagactcggcagcatctccaggtcaacactgacctgggggggagcctcccgatctgtgttccagcgtgtcaagagtgctacagagagaaagaacgtgtcttcctagacatcccaagggagactgagtgggtcaacacctccaccacttcagacaaggcccaaacctacttctccacctggtgtgatctcccaagagtgtccaccctcccctcactagaccagaaggacgcaacctgtctgatggggaggtccatggcaactcaaaaatctactccgggggaatccatcaccaagttctacagcacagccccagacacccatcgacttctccatcctgtgaccttggtcctgggctatgcagaaggaacaattttttcttacatgaaaagtcagcacaacattcatgggtccctttaccatccgaacatcgaagagatcgaaccagctcttgagaagtatgtgatcgacacgaagacatcccataccaagcacctgggatatctgtttcaagatgccgactccctccaggaactgctagagacaggaatgacaccatacattccccgatccattcctctcacgatcactgaactgacaagtgcatgctgcatgacacttgccaaagcaatctccattgtcctcagaaccggagtgaccattccattgatgccagaaaatggatacggagaaaacgatatccaagtggcaagactcaccgccaactccctttcccgactcatgcccagggggaggattcagctggtctacctggactgtgacctgacctcgcagatgaccgcgtgggtgccaaccagccagccgagcgtcctgggatctgtcaacttccacatcgagggggttaccatctccctcacagcaacagagatgagagtgggacaagaaagctgggaagagagaaagtggacatgttcaaacaacagacacatcatcgcaaagggggtcaagaccaaatctctgttcatacatcagagcgtccccgagatcatcacacacccgcccgacctcattgtggtgataggagggggcctagggggctgtgtagtgccctaccttcaaaagtggagaggtcccaaagtaatcttctgcactctctttgatgaacgagagcggatctcggaggacggagacctgatcattcccccggagctcctagttcgaggactcgctgggagaatgattgagaaagaactcctggaggctgagatgtgtgacatcacggtcaaggggaatagggacctcctcatcaaagtagtgcagaaatgggttcagccgaacgaacatgtccttctcattgatgagattgaaaataggggggatcaagagtcagtactccaatcttccatcagtgagcttctgacaagaatggacagcgtgtgcaacctcacttctgttcacaccatccgagagaccggacctcgacaattcgcccagagggtgaacacgattaggagaggcagaaggacagcaacactccactggaaccaatacaaccgaagagaccaggtggaggccctcctacttgttgagagccacaccaggaagaccgaactgcacgtcacatcatccgccgtacaggcggcattcagaaagatagacgagaagctcgagtcggagagtcgactagagcacagcaaatggtcactccctgaactccctccaagggagaaagacatcctacttggatatgtggcatctgtctttttaaagctagggctggttgtgacagacagacacatgtcggctgccgcgctcatcactctcctcgaagaagctgggccaaaaatgatatcctgggataagaagatggaacatcgaacctgggcctcctcggatgccatcactgagaaaggaatcacccaggaccagatcttttcccttctctgctttgcatgggcccttaggggactaaaatcaggggactgggaacacaacgcagacgccatcattctccaggatgtccacatcgacacaggtccgaggctctgtcagatgggcgagtctcccaagaggacatttgcatccttcaggctgcacaacaccaaaaaggcggaggatctcaaggggtacttaggtgctctgctccacctggagtccttctttccatttggagaacagtgacgatcgccccgctcatcaattagattgaaaaaaactatgacctgggttcaatgaccacgtgaaccctcgtgggcaccatgtcccagacctcccgcctcggattt

SEQ ID NO: 28 - protein, 404 aa, Viral hemorrhagic septicemia virus
MDGGLRAAFSGLNDVRIDPTGGEGRVLVPGEVELIVYVGGFSADDGKVIVDALSALGGPQTVQALSVLLSYVLQGNKQEDLEAKCKVLTDMGFRVTQSTRATGIDAGILMPMRELALAVNNDSLMDIVKGTLMTCSLLTKYSVDKMIKYITKKLGDLADTQGIGELQHFTADKAAIRKLAGCVRPGQKITKALYAFILTEIADPTTQSRARAMGALRLNGTGMTMIGLFTQAANNLGIAPAKLLEDLCMESLVESARRIIQLMRQVSEAKSIQERYAIMMSRMLGESYYKSYGLNDNSKISYILSQISGKYAVDSLEGLEGIKVTEKFREFAELVAEVLVDKYEKIGEDSTEVSDVIREAARQHARRTSAKPEPKARNFRSPTGRGKEQDTEESDDEDYPEDSD

SEQ ID NO: 29 - protein, 222 aa, Viral hemorrhagic septicemia virus
MADIEMSESLVLSHGSLADLDRKLDNAPKDTRSALFSSTSGFTKQKSSPKKKPNPTTLEELIGHFVSEDLQLDATKAFGQLLRRIKMSHQEELTQHLEKVNGENRAKMGALLESQKENGKKTDNILSILISMRGEGAENASKKPKVLDGDQVRNERALGFNRGLTTAAIAMKKFKLEDPLALCKGSVKRAALSAMEKEEYDGERETYSTVSKAIKAELDKLE

SEQ ID NO: 30 - protein, 201 aa, Viral hemorrhagic septicemia virus
MTLFKRKRTILVPPPHITSNDEDRVSTILTEGTLTITGPPPGNQVDKICMAMKLARAILCEDQHPAFNPLVYLFQSAMIFGETSEKIDFGTRSKTLITSFKIAEAKAIYLESSPVRSRIEAKKYTTPIRHGSVTYYGPFVFADDHVGGKGHREKLGALCGFLQSGPYGQAKDYYNRAVEEEMGILPRDPKRRSGASSAQPR

SEQ ID NO: 31 - protein, 507 aa, Viral hemorrhagic septicemia virus
MEWNTFFLVILIILIKSTTSQITQRPPIENISTYHADWDTPLYTHPSNCREDSFVPIRPAQLRCPHEFEDINKGLVSVPTQIIHLPLSVTSVSAVASGHYLHRVTYRVTCSTSFFGGQTIEKTILEAKLSRQEAANEASKDHEYPFFPEPSCIWMKNNVHKDITHYYKTPKTVSVDLYSRKFLNPDFIEGVCTTSPCQTHWQGVYWVGATPTAHCPTSETLEGHLFTRTHDHRVVKAIVAGHHPWGLTMACTVTFCGTDWIKTDLGDLIKVVGQGGETKLTPKKCVNADIQMRGATDDFSYLNHLITNMAQRTECLDAHSDITASGKISSFLLSKFRPSHPGPGKAHYLLDGLIMRGDCDYEAVVSINYNSAQYKTVNNTWKSWKRIDNNTDGYDGMIFGDKLIIPDIEKYQSIYDSGMLVQRNLVEVPHLSIVFVSNTSDLSTNHIHTNLIPSDWSFNWSLWPSLSGMGVVGGAFLLLVLCCCCKASPPIPNYGIPMQQFSRNQMV

SEQ ID NO: 32 - protein, 122 aa, Viral hemorrhagic septicemia virus
MTTQLALDTVSFSPLVLREMITHRLTFDPSNYLNCDIDRSDVSTMSFFETTLPRILDDLRACPRLPYLHVLDMRISLLERTHYMFKNVPSSPATTGRPTDPGLIIISCADVGVLTNGSGFTS

SEQ ID NO: 33 - protein, 1984 aa, Viral hemorrhagic septicemia virus
MEMFELDREVHQERLPSECSLNSPLNLSLSLQLFGRLTPKTEHIRYQAGRIKRWLVKQYQLVHLRELEIDSTRIQGYLIPHLLKTQSNELGSSVIKNWGMVSKYYLSLGYTLPPKDKFDFREVAPYWNLASQLREVTLESQKVDTRGKEKRKLYQVEDVEFEFKEGVVVIRAGLDGLLNEFLGGEKLGAVTYVEYLAFFKIINQRAQALLLTAICQALEPDLVPPCSGILSVYAEVDSVLRRAGQSAIDLLKLWEPLVLTKLGDVLGDRFGLEDDFKDTIRGEANKLAKKLHVTRSYKRMMKTLDQETRAQALFQYFGLFKHFAYPRVYSRETIEAIQEVSDKPSDSSPLNYLSDQCKIREEFYIRYTKAYHRAPAMNLGQLGQGSYLRQVLEAGKIPNAKNALYSLLEWFFVRFEKSIEWPLSDTLSTFLSDKAITQNRDIWYDGGSSGRDTTEKRLLLKFIKENEDSVEKVILKADEIYDKEADQIIALKVKEMELKIKGRGFGLMAFKPRLLQVLRESIAKKTSKLFPEITMTFSDLELKKKKFQLSRKSDDRRGYIHISKSLDINKFCTSQRQFNSLAVFQSLDELLGTDQLFTRVHEIFEKTWIVDGSASDPPDLVTFKARYEEALALGIEAPHVWADGAFSGLMGGIEGLCQYVWTICLLLRVERVLAVTQLTHFVMAQGDNVIINLIIPVEVDRVGGVVEGERARIQRISKDIDSALERELLRSGLTLKIEETLTSENLSIYGKDLHCPQHLTLAVKKAGSASIISSEQYQDVPTFLSGLGTGMETISECVNNKVSAHLFGVILGAAGWKSLAQRQTWKGWEYPFQNETSRRQVRSQGILLQKGESTMVHKEPEANPEKRTIELLLVSSLFGSALGMLPFPTPIDLEKRGVGDYVTHRLSIVKMALVSKKLPNRMIEMIVSTMNLPLSREQDLTKLFDSPFSLNLATEEDAASVIKRLARGTLRGLDIKNKKLADHIATMDQGITQIDEALASADTINPRIAYQFRNITDQKESEMFVTKFATAKTMRMVALSSSQDVSVVGLLNKRSQAKEIYTIWRTQRKGETLWTCSTQQAKKLRDRSWGKNIIGVTSPSPLEAILFKLIDPISWEEEKEAHHFTIHYYLSKPSLSSKTAHTTRGPLVPYFGTQTKPLIAKAYMELKGNPRTNKALQLLSMRETMIKAGSNLDKLLLSLCSNALDIDVNSLPSLQAQEEASAGEGVRGGIKESMSPVGPDNLYTHITHKVFERQWLSEFHVNIADFIIWGITKTRQHLQVNTDLGGSLPICVPACQECYREKERVFLDIPRETEWVNTSTTSDKAQTYFSTWCDLPRVSTLPSLDQKDATCLMGRSMATQKSTPGESITKFYSTAPDTHRLLHPVTLVLGYAEGTIFSYMKSQHNIHGSLYHPNIEEIEPALEKYVIDTKTSHTKHLGYLFQDADSLQELLETGMTPYIPRSIPLTITELTSACCMTLAKAISIVLRTGVTIPLMPENGYGENDIQVARLTANSLSRLMPRGRIQLVYLDCDLTSQMTAWVPTSQPSVLGSVNFHIEGVTISLTATEMRVGQESWEERKWTCSNNRHIIAKGVKTKSLFIHQSVPEIITHPPDLIVVIGGGLGGCVVPYLQKWRGPKVIFCTLFDERERISEDGDLIIPPELLVRGLAGRMIEKELLEAEMCDITVKGNRDLLIKVVQKWVQPNEHVLLIDEIENRGDQESVLQSSISELLTRMDSVCNLTSVHTIRETGPRQFAQRVNTIRRGRRTATLHWNQYNRRDQVEALLLVESHTRKTELHVTSSAVQAAFRKIDEKLESESRLEHSKWSLPELPPREKDILLGYVASVFLKLGLVVTDRHMSAAALITLLEEAGPKMISWDKKMEHRTWASSDAITEKGITQDQIFSLLCFAWALRGLKSGDWEHNADAIILQDVHIDTGPRLCQMGESPKRTFASFRLHNTKKAEDLKGYLGALLHLESFFPFGEQ